(19) United States
(12) Patent Application Publication
(10) Pub. No.: US 2023/0399335 A1
(43) Pub. Date: Dec. 14, 2023

(54) PROCESS FOR THE PREPARATION OF ASENAPINE IN THE FORM OF ITS FREE BASE

(71) Applicant: LTS LOHMANN Therapie-Systeme AG, ANDERNACH (DE)

(72) Inventors: René RIETSCHER, ANDERNACH (DE); René EIFLER, Koblenz (DE); Olga BOURQUAIN, Dürrholz (DE)

(21) Appl. No.: 18/010,922

(22) PCT Filed: Jun. 4, 2021
(86) PCT No.: PCT/EP2021/065062

§ 371 Date: Dec. 16, 2022

(30) Foreign Application Priority Data
Jun. 19, 2020 (DE) 10 2020 207 642.1

(51) Int. Cl. C07D 491/044; A61K 9/70

(52) U.S. Cl. CPC C07D 491/044; A61K 9/7038

(57) ABSTRACT

The present invention relates to a process for the preparation of asenapine in the form of its free base, a process for the preparation of an active ingredient-containing layer for use in a transdermal therapeutic system, asenapine in the form of its free base, obtainable by said process for the preparation of asenapine in the form of its free base, an active ingredient-containing layer for use in a transdermal therapeutic system, obtainable by said process for the preparation of an active ingredient-containing layer and a transdermal therapeutic system containing such an active ingredient-containing layer.

### TECHNICAL FIELD OF THE INVENTION

The present invention relates to a process for the preparation of asenapine in the form of its free base, a process for the preparation of an active ingredient-containing layer for use in a transdermal therapeutic system (TTS), asenapine in the form of its free base in high purity, an active ingredient-containing layer for use in a transdermal therapeutic system, and a transdermal therapeutic system.

### BACKGROUND OF THE INVENTION

The active ingredient asenapine (3aRS,12bRS)-rel-5-chloro-2,3,3a,12b-tetrahydro-2-methyl-1H-dibenz[2,3:6,7]oxepin [4,5-c]pyrrole) is an atypical antipsychotic of the dibenzo-oxepin pyrrole family, the tetracyclic structure of which is unrelated to that of other antipsychotics such as olanzapine, quetiapine or clozapine (tricyclic structure), risperidone, ziprasidone or aripiprazole (bicyclic structure). Asenapine is an antagonist at the dopamine D2 and serotonin 5-HT2A receptors with a high affinity for the latter, and was developed by Schering-Plow/Organon for the treatment of schizophrenia and a bipolar disorder associated acute mania.

Asenapine is currently commercially available in the form of sublingual tablets under the brand name Sycrest (Swissmedic) and Saphris (Schering-Plow) which are administered in dose strengths of 2.5 mg, 5 mg or 10 mg twice daily (bid).

The sublingual route of administration avoids the first-pass metabolism of oral administration in order to increase bioavailability, which is 35% when taken sublingually, and <2% when taken orally. However, the sublingual administration is accompanied by a bitter or unpleasant taste, as well as numbness of the tongue/oral mucosa, nausea and headache caused by a local anesthetic effect. In addition, eating, drinking and smoking is prohibited for 10 minutes immediately after sublingual dosing. These inconveniences can lead to worsened behavior by the patient with regard to correct intake and improper administration, such as dose reduction, dose skipping, irregular active ingredient intake or complete abandonment of the intended asenapine intake. Sublingual administration is also difficult to supervise in institutionalized psychiatric patients, and may also be unsuitable for children, the elderly, and other patients with difficulty swallowing, or for those unable to self-medicate.

The disadvantages of sublingual administration can be avoided by transdermal administration of asenapine. For the transdermal administration of asenapine, so-called transdermal therapeutic systems (TTS) are used, which contain asenapine and can release asenapine in contact with the skin, which enables the transdermal administration of asenapine.

Known such transdermal therapeutic systems include asenapine maleate or asenapine in the form of its free base. The active ingredient in the form of its free base usually has the advantage of improved skin permeation compared to the active ingredient in protonated form. Accordingly, there is a need for asenapine in the form of its free base for the preparation of transdermal therapeutic systems with favorable skin permeation. Since transdermal therapeutic systems are pharmaceutical products, asenapine in the form of its free base must also be of high purity. However, asenapine in the form of its free base in high purity is only available to a limited extent commercially. In order to ensure permanent and stable commercial production of transdermal therapeutic systems containing asenapine in the form of its free base, a high availability of asenapine in the form of its free base with a high degree of purity is of crucial importance. In contrast, asenapine maleate, a maleic acid addition salt of asenapine, is used commercially and in large quantities which can be produced under qualitative conditions of good manufacturing practice. Accordingly, it could be advantageous to the commercial preparation of transdermal therapeutic systems containing asenapine in the form of its free base, if asenapine in the form of its free base could be prepared in sufficient quantities in high purity from asenapine maleate.

Accordingly, there is a need to provide a preparation process by means of which asenapine in the form of its free base can be prepared in high purity and at high conversion, i.e. in large quantities, from asenapine maleate.

### OBJECT AND SUMMARY OF THE INVENTION

It is a object of the present invention to provide a preparation process of asenapine in the form of its free base using asenapine maleate which overcomes the above-mentioned disadvantages in terms of the availability of asenapine in the form of its free base in high purity.

Therefore, it is a object of the present invention to provide a preparation process by means of which asenapine in the form of its free base can be produced at high conversions from asenapine maleate, wherein asenapine in the form of its free base has high purity.

These and other objects are achieved by the present invention which, according to a first aspect, relates to a process for the preparation of asenapine in the form of its free base, wherein the process comprises the steps of:
1) providing a reaction mixture comprising asenapine maleate and an alkali metal silicate in a solvent, wherein the reaction mixture contains the alkali metal silicate in dispersed form; and
2) reacting asenapine maleate with the alkali metal silicate in the reaction mixture provided in step 1) in order to obtain a product mixture which contains dissolved asenapine in the form of its free base and alkali metal maleate in dispersed form.

According to certain embodiments of the present invention, the process further comprises the step of:
3) isolating a solution containing asenapine in the form of its free base from the product mixture obtained in step 2), wherein the isolation is preferably carried out by means of filtration.

According to a second aspect, the present invention relates to a process for the preparation of an active ingredient-containing layer for use in a transdermal therapeutic system, wherein said process comprises the following steps of:
i) preparing asenapine in the form of its free base by means of the process according to the first aspect;
ii) combining at least the asenapine in the form of its free base obtained in step i) and a polymer in a further solvent in order to obtain a coating composition, wherein the asenapine in the form of its free base obtained in step i) and used in step ii) is preferably contained in the solvent used in step ii), and is more preferably present in a solution isolated according to step 3) according to certain embodiments of the first aspect of the present invention;
iii) coating the coating composition on a back layer, a peelable film or an intermediate film; and
iv) drying the coated coating composition to form the active ingredient-containing layer.

According to a third aspect, the present invention relates to asenapine in the form of its free base, obtainable by a process according to the first aspect of the present invention wherein the asenapine in the form of its free base has a purity of 99.0% or more, preferably 99.5% or more, particularly preferably 99.95% or more, wherein the purity is determined by means of HPLC, and and wherein the asenapine in the form of its free base is preferably present in a solvent and particularly preferably in a solution isolated according to step 3) according to certain embodiments of the first aspect of the present invention.

According to a fourth aspect, the present invention relates to an active ingredient-containing layer for use in a transdermal therapeutic system, obtainable by a process according to the second aspect of the present invention, wherein the asenapine in the form of its free base in the active ingredient-containing layer has a purity of 99.0% or more, preferably 99.5% or more, particularly preferably 99.95% or more, wherein the purity is determined by means of HPLC.

In a fifth aspect the present invention relates to a transdermal therapeutic system containing an active ingredient-containing layer according to the fourth aspect of the invention.

For the purpose of the invention, the term “asenapine in the form of its free base” refers to the chemical compound which is identified by the chemical nomenclature “(3aRS,12bRS)-rel-5-chloro-2,3,3a,12b-tetrahydro-2-methyl-1H-dibenz [2,3:6,7]oxepin[4,5-c]” pyrroler and is represented by the following formula (I):

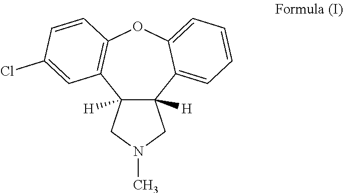 [SMILES: CN1C[C@H]2c3ccccc3Oc3ccc(Cl)cc3[C@@H]2C1]

For the purpose of the invention, the term “asenapine maleate” refers to a compound in the form of an acid addition salt of asenapine in the form of its free base and maleic acid.

For the purpose of the present invention, an “alkali metal silicate” can be understood to mean any salt composed of an alkali metal cation and a silicate anion. The cations of an alkali metal silicate can consist exclusively of alkali metal cations, as is the case, for example, with sodium metasilicate. Alkali metal cations can, for example, be selected from the group consisting of lithium, sodium, potassium, cesium or combinations thereof. Furthermore, the term “alkali metal silicate” also includes “heterogeneous alkali metal silicates”, wherein such a heterogeneous alkali metal silicate contains alkali metal cations and additionally higher-valent cations which are, for example, selected from the group consisting of calcium, magnesium, aluminum, or combinations thereof. Examples are potassium aluminum disilicates or sodium aluminosilicates, such as, for example, AlNa₁₂SiO₅. Silicate anions can, for example, be selected from the group consisting of metasilicate, trisilicate or combinations thereof. The alkali metal cations and the silicate anions can be combined with one another as desired.

For the purpose of this invention, the term “providing a reaction mixture” refers to mixing any substances, which can be used to provide the reaction mixture, such as solvent (e.g., ethanol and water), asenapine maleate, antioxidants, and alkali metal silicate in a predefined amount. The reaction mixture can also contain a protective gas atmosphere, in particular comprising argon and/or nitrogen.

Accordingly, for the purpose of the invention, the term “reaction mixture” is understood to mean a mixture of all substances which which are used for the reaction of asenapine maleate with the alkali metal silicate to form asenapine in the form of its free base; regardless of whether these substances react chemically during the reaction, such as, for example, asenapine maleate and the alkali metal silicate, or remain chemically unchanged, such as, for example, the solvent. The reaction mixture is completely ready when no further substances are added. The “reacting asenapine maleate” therefore follows directly after “providing the reaction mixture”, i.e. from the point in time at which the last substance, for example the alkali metal metasilicate, was added to the reaction mixture. Therefore, the step of reacting is carried out immediately following the step of providing the reaction mixture. The reaction mixture contains at least asenapine maleate as well as an alkali metal silicate in dispersed form in a solvent.

For the purpose of this invention, the term “solvent” in connection with the reaction mixture as well as with the product mixture refers to a liquid which comprises, for example, water and/or an alcohol, wherein the alcohol can be selected from the group consisting of methanol, ethanol, 1-propanol and/or 2-Propanol, and is used, among other things, so that at least a portion of the asenapine maleate is present in dissolved form. In the solvent both solids and dissolved substances can be present. For example, asenapine maleate can be completely dissolved or present to some extent as solid and to some extent as dissolved substance in the solvent of the reaction mixture. The alkali metal silicate, in particular, is largely present as a solid in the solvent and can be partially dissolved in the solvent.

For the purpose of this invention, “reacting” is understood to mean the reaction between asenapine maleate and the alkali metal silicate in the solvent, forming asenapine in the form of its free base in dissolved form as well as alkali metal maleate in dispersed form by this reaction, each of which are included in a product mixture. Reacting can be carried out using a protective gas atmosphere, in particular comprising argon and/or nitrogen.

For the purpose of this invention, the “product mixture” contains dissolved asenapine in the form of its free base and the alkali metal maleate in dispersed form in the same solvent that was also used for the reaction mixture. In addition, depending on the conversion, the product mixture can contain dissolved asenapine maleate and asenapine maleate in dispersed form. Without wishing to be bound by theory, it is believed that the product mixture also contains silicon dioxide in dispersed form, which is formed by the protonation of the silicate anion of the alkali metal silicate and a subsequent elimination of water of the resulting free silicic acid. Furthermore, the product mixture can contain other substances which were also present in the reaction mixture, such as, for example, antioxidants.

For the purpose of the present invention, the term “dispersed form” is understood to mean, that a substance can be present as a solid in the solvent of the reaction mixture or of the product mixture, for example, in an amount of 1% by mass or more, 10% by mass or more, 20% by mass or more, 50% by mass or more, 60% by mass or more, 70% by mass or more, 80% by mass or more, 90% by mass or more, or 95% by mass or more; or completely, i.e. about 100% by mass, based on the total mass of the substance used. The fraction of the substance, which in this case, is not present as a solid, but is present in dissolved form is, accordingly, for example, 99% by mass or less, 90% by mass or less, 80% by mass or less, 50% by mass or less, 40% by mass or less, 30% by mass or less, 20% by mass or less, 10% by mass or less, or 5% by mass or less or about 0% by mass (when completely present as a solid), based on the total mass of the substance used. Such a substance can include, for example, the alkali metal silicate in the reaction mixture and the alkali metal maleate in the product mixture. During the reaction, the fractions defined above can change dynamically due to the reaction and the associated variation in solubility.

For the purpose of the present invention “dissolved asenapine in the form of its free base” is understood to mean that asenapine in the form of its free base is present in a dissolved form in the solvent of the product mixture and, for example, can not be separated from the solvent by filtration with a blue-band filter or centrifugation for 15 minutes and 15,000 rpm at room temperature.

For the purpose of the present invention, “isolating a solution containing asenapine in the form of its free base from the product mixture” is understood to mean that solids present in the product mixture, such as, for example, alkali metal maleate and silicon dioxide, are separated. Said isolating results in a solids-free solution which contains the solvent and dissolved asenapine in the form of its free base. Such isolation can be carried out by means of filtration, a solid phase extraction, a sedimentation, a decanting, or a centrifugation.

For the purpose of the present invention, the term “particle diameter” denotes either a d50 particle diameter or a d80 particle diameter of the alkali metal silicate. In this case, the d50 diameter can be less than 125 μm. Furthermore, the d80 diameter can be less than 200 μm. In this case, when determining the particle diameter, elutriation by means of a suitable screening machine, or screening by means of a sieve shaker, preferably by means of a sieve shaker, with corresponding test sieves can be used. Preferably, the determination is done according to DIN 66165. Furthermore, for the determination of the d50 particle diameter or the d80 particle diameter, a particle size distribution based on the mass of the particles, the volumes of the particles, or based on the number of particles can be determined. Preferably, the particle size distribution is determined based on the mass of the particles.

For the purpose of the present invention, the term “ground” is understood to mean that particles, in particular alkali metal silicate particles, are comminuted either in a mortar with a pestle or by means of a ball mill, a hammer mill, or by means of ultrasound.

For the purpose of the present invention, the term “total mass of the reaction mixture” denotes the sum of the masses weighed of all substances, which were used for providing the reaction mixture.

For the purpose of the invention, the term “conversion” denotes the mass fraction of dissolved asenapine in the form of its free base based on the total mass of asenapine dissolved in the product mixture. This “total mass of asenapine” consists of dissolved asenapine in the form of its free base and dissolved (i.e., unreacted) asenapine maleate. To determine the conversion, a sample is taken from the product mixture which contains a solids fraction. This solids fraction is removed by centrifugation for 15 minutes at room temperature and 15,000 rpm, so that a centrifuged solids-free solution can be taken out. The conversion is then determined based on the data of a quantitative HPLC analysis, which will be explained in detail below. In particular, the integrated areas of the total mass of asenapine and the maleic acid (derived from dissolved asenapine maleate) are obtained from the chromatogram. Using an appropriate calibration, which will be explained in detail below, the masses of the asenapine maleate and total mass of asenapine can be obtained from the integrated areas of the chromatogram. The mass fraction of the asenapine originating from asenapine maleate based on the total mass of asenapine can then be calculated using the mass of maleic acid and subtracted from the total mass of asenapine, whereby the mass (and also the mass fraction) of the dissolved asenapine in the form of its free base can be obtained. In this case, a conversion of 0% is understood to mean that no conversion of asenapine maleate to asenapine in the form of its free base was taking place, whereas, a conversion of 100% determined in such a way is understood to mean that asenapine maleate was completely converted to asenapine in the form of its free base.

For the purpose of the invention, the “purity of asenapine in the form of its free base in the product mixture” is defined via the “sum of the degradation products”, wherein the sum of the degradation products is the sum of the relative percentages of the integrated areas per degradation product determined in the HPLC chromatogram based on the calculated area of asenapine in the form of its free base. The smaller the sum of the degradation products, the higher the purity of the asenapine in the form of its free base. For example, a sum of the degradation products of 0.05% results in a purity of asenapine in the form of its free base of 99.95%. The purity can therefore be calculated using the formula “100%−sum of degradation products”. In this case, the centrifuged solution described above is used as a basis for the HPLC-sample. The degradation products can form during the conversion. In the present case, possible degradation products of asenapine include asenapine N-oxide (cis), asenapine N-oxide (trans), deschloro asenapine and tetrahydroasenapine, for example.

For the purpose of the present invention, the term “further solvent” is not used for a solvent contained in the reaction mixture or in the product mixture, but for a further solvent, which is used for the preparation of an active ingredient-containing layer according to the second aspect of the invention. Therefore, the term “further solvent” refers to any liquid substance, which is preferably a volatile organic liquid such as methanol, ethanol, isopropanol, acetone, ethyl acetate, dichloromethane, hexane, n-heptane, toluene and mixtures thereof.

For the purpose of the present invention, the term “antioxidants” is understood to mean substances that are suitable to reduce the degradation of asenapine in the form of its free base to form degradation products, for example, the above-defined degradation products. Examples of the antioxidants are sodium metabisulfite, α-tocopherol and ascorbyl palmitate.

For the purpose of the present invention, the “purity of asenapine in the form of its free base in the active ingredient-containing layer” is determined using quantitative HPLC analysis, which is presented in detail in the detailed description section.

For the purpose of the present invention, an “active ingredient-containing layer” can be a matrix layer, for example.

For the purpose of this invention, the term “transdermal therapeutic system” (TTS) refers to a system through which the active ingredient (asenapine) is administered to the systemic circulation by means of transdermal delivery, and refers to the entire individual dosage unit that is applied on a patient's skin, and which comprises a therapeutically effective amount of asenapine (in the form of its free base) in a self-adhesive layer structure and optionally an additional adhesive top layer on the asenapine-containing (in the form of its free base) self-adhesive layer structure. The self-adhesive layer structure can be placed on a peelable film (a detachable protective layer), and therefore the TTS can further comprise a peelable film. For the purpose of this invention, the term “TTS” refers in particular to a system which provides passive transdermal delivery which excludes active transport as in methods including iontophoresis or microperforation.

There are two main types of TTS, which make use of passive active ingredient delivery, namely TTS of the matrix type and TTS of the reservoir type. In TTS of the matrix type, the active ingredient is incorporated in a matrix, while in TTS of the reservoir type, the active ingredient is incorporated in a liquid or semi-liquid reservoir. The release of an active ingredient in a TTS of the matrix type is primarily controlled by the matrix, which contains the active ingredient itself. In contrast, a TTS of the reservoir type requires a rate controlling membrane that controls active ingredient release. TTS of the matrix type are advantageous in that, compared to TTS of the reservoir type, no rate-determining membranes are usually necessary and no sudden dose release due to a ruptured membrane can occur. In summary, transdermal therapeutic systems (TTS) of the matrix type are less complex to prepare and are simple and convenient to use by the patients.

For the purpose of this invention, the term “TTS of the matrix type” refers to a system or structure wherein the active ingredient is homogeneously dissolved and/or dispersed in a polymeric carrier, i.e. the matrix, which forms the matrix layer together with the active ingredient and optionally the remaining ingredients. In such a system the matrix layer controls the release of the active ingredient from the TTS. A TTS of the matrix type can also include a rate controlling membrane.

TTS with a rate controlling membrane and a liquid or semi-liquid reservoir containing the active ingredient, wherein the active ingredient release is controlled by the TTS by the rate controlling membrane are referred to with the term “TTS of the reservoir type”. For the purpose of the invention, TTS of the reservoir type are not to be understood as of the matrix type. In particular, for the purpose of this invention, microreservoir systems (two-phase systems which have an inner active ingredient-containing phase in an outer matrix phase) which are considered in the field as a blend between a TTS of the matrix type and a TTS of the reservoir type, are considered to be of the matrix type for the purpose of the invention. TTS of the matrix type can in particular be in the form of a TTS of the “active ingredient-in-adhesive” type, which relate to a system in which the active ingredient is homogeneously dissolved and/or dispersed in a pressure-sensitive adhesive matrix.

For the purpose of this invention, the term “matrix layer” refers to any layer which contains the active ingredient homogeneously dissolved and/or dispersed in a polymeric carrier. In a TTS of the matrix type, there is typically a matrix layer as the active ingredient-containing layer. A TTS of the reservoir type can contain, in addition to the reservoir layer and a rate controlling membrane, an additional adhesive layer which serves as a skin contact layer. In such a TTS of the reservoir type, the additional adhesive layer is often prepared as an active ingredient-free layer. However, as a result of the concentration gradient, the active ingredient will move over time from the reservoir to the additional adhesive layer until equilibrium is reached. Therefore, in such a TTS of the reservoir type, the additional adhesive layer contains the active ingredient after a certain time of establishment of equilibrium, and is to be regarded as a matrix layer for the purpose of the present invention.

The matrix layer is the final, solidified layer, which is obtained, for example, by coating and drying the solvent-containing coating composition. The matrix layer can also be made by laminating two or more such solidified layers (e.g., dried layers) of the same composition to provide the desired grammage. The matrix layer can be self-adhesive (in the form of a pressure-sensitive adhesive matrix) or the TTS can comprise an additional skin contact layer of a pressure-sensitive adhesive to provide sufficient tack. In particular, the matrix layer is a pressure-sensitive adhesive matrix.

For the purpose of this invention, the term “grammage” refers to the dry weight of a specific layer, for example the matrix layer, given in g/m². The grammage values are subject to an error tolerance of ±10%, preferably ±7.5%, due to preparation fluctuations.

For the purpose of this invention, the term “asenapine-containing (in the form of its free base) self-adhesive layer structure” or “self-adhesive layer structure containing a therapeutically effective amount of asenapine in the form of its free base” refers to a structure containing the active ingredient which structure provides the release area for asenapine during administration. The adhesive top layer adds to the overall size of the TTS but does not add to the release area. The asenapine-containing (in the form of its free base) self-adhesive layer structure comprises a back layer and at least one asenapine-containing layer.

For the purpose of this invention, the term “pressure-sensitive adhesive” refers to a material that sticks in particular to finger pressure, is permanently tacky, exerts a strong holding force and should be removable from smooth surfaces without leaving any residue. A pressure-sensitive adhesive layer, when it is in contact with the skin, is “self-adhesive”, i.e. provides a bond strength to the skin so that typically no further support is required for application to the skin. A “self-adhesive” layer structure includes a pressure-sensitive adhesive layer for skin contact, which can be provided in the form of a pressure-sensitive adhesive matrix or in the form of an additional layer, i.e. a pressure-sensitive skin-contact adhesive layer. An adhesive top layer can still be used to improve the bond strength.

Unless otherwise stated, “%” refers to mass percent, which is also abbreviated as “% by mass”.

For the purpose of this invention, the term “polymer” refers to any substance consisting of so-called repeating units, which are obtained by polymerizing one or more monomers, and includes homopolymers, which consist of one monomer type, and copolymers, which consist of two or more monomer types. Polymers can be of any architecture, such as linear polymers, star-shaped polymers, comb polymers, brush-shaped polymers, of any monomer arrangement in the case of copolymers, for example alternating, random, block copolymers, or graft polymers. The minimum molecular weight differs depending on the type of polymer and is known to the person skilled in the art. Polymers can, for example, have a molecular weight above 2,000, preferably above 5,000 and more preferably above 10,000 Daltons. Accordingly, compounds with a molecular weight below 2,000, preferably below 5,000 and more preferably below 10,000 Daltons are usually referred to as oligomers.

For the purpose of this invention, the term “functional groups” refers to hydroxyl and carboxylic acid groups.

For the purpose of this invention, the term “crosslinking agent” refers to a substance which is capable of crosslinking functional groups contained in the polymer.

For the purpose of this invention, the term “adhesive top layer” refers to a self-adhesive layer that is free of active ingredient and larger in area than the active ingredient-containing structure, and that provides additional area adhering to the skin, but not an area of release of the active ingredient. It improves the overall adhesive properties of the TTS. The adhesive top layer comprises a back layer and an adhesive layer.

For the purpose of this invention, the term “back layer” refers to a layer which, for example, carries the asenapine-containing active ingredient-containing layer or which forms the back layer of the adhesive top layer. At least one back layer in the TTS, and usually the back layer of the asenapine-containing layer, is occlusive, i.e. essentially impermeable to the active ingredient contained in the layer during storage and administration, and thus prevents active ingredient loss or cross-contamination in accordance with the licensing regulations.

For the purpose of this invention, the term “coating composition” refers to a composition comprising all components of the active ingredient-containing layer, which is, in particular, a matrix layer, in a further solvent and which can be coated onto a back layer or a peelable film in order to form the active ingredient-containing layer, which is, for example, a matrix layer.

For the purpose of this invention, the term “room temperature” refers to an unchanged temperature which is found inside the laboratory where the experiments are carried out, and is usually between 15 and 35° C., preferably about 18 to 25° C.

For the purpose of this invention, and unless otherwise stated, the term “about” refers to an amount that is ±10% of the disclosed amount. In some embodiments, the term “about” refers to an amount that is ±5% of the disclosed amount. In some embodiments, the term “about” refers to an amount that is ±2% of the disclosed amount.

### DETAILED DESCRIPTION

### Process for the Preparation of Asenapine in the Form if its Free Base

According to a first aspect, the present invention relates to a process for the preparation of asenapine in the form of its free base.

The process comprises first a step 1) of providing a reaction mixture wherein asenapine maleate as well as alkali metal silicate are used in a solvent. This produces a reaction mixture in which asenapine maleate can be present in dispersed form, i.e. partly dissolved and partly as a solid, or completely dissolved in the solvent. The solubility of the asenapine maleate depends in particular on the type and the mass of the solvent used and on the mass of the asenapine maleate used. In any event, the alkali metal silicate is present as part of the reaction mixture in dispersed form in the solvent. Here, the asenapine maleate can be completely or partially present as a solid in the solvent. The asenapine maleate can be present in the reaction mixture as a solid in an amount of 0% by mass, 10% by mass or more, 30% by mass or more, 50% by mass or more, or 70% by mass or more, based on the total mass of the asenapine maleate used. Correspondingly, asenapine maleate can be present in dissolved form in the solvent in an amount of 100% by mass, 90% by mass or less, 70% by mass or less, 50% by mass or less, or 30% by mass or less, based on the total mass of the asenapine maleate used. The alkali metal silicate can essentially be present as a solid in the solvent of the reaction mixture, for example in an amount of 50% by mass or more, 60% by mass or more, 70% by mass or more, 80% by mass or more, 90% by mass or more, or 95% by mass or more; or completely, that is, to about 100% by mass, based on the total mass of the alkali metal silicate used. It was found, surprisingly, that the use of an alkali metal silicate, i.e. using alkali metal cations, in particular potassium cations and/or sodium cations, is important because metal silicates, that only contain higher-valent metal cations, such as, for example, magnesium cations, calcium cations or aluminum cations, do not lead to a significant conversion of asenapine maleate. The fraction of the alkali metal silicate which is not present in the solvent as a solid but in dissolved form is, for example, 50% by mass or less, 40% by mass or less, 30% by mass or less, 20% by mass or less, 10% by mass or less, or 5% by mass or less, or 1% by mass or less, based on the total mass of the alkali metal silicate used. In other words, the reaction mixture is a dispersion which contains fractions of a suspension and fractions of a solution.

After the reaction mixture has been completely provided, i.e. no further substances are added, in a second step 2) reacting at a specific temperature, for example at room temperature and for example with stirring, is carried out. After the reaction, a product mixture is formed which contains dissolved asenapine in the form of its free base and alkali metal maleate in dispersed form (e.g., with a solids fraction of about 100% by mass, based on the total mass of the alkali metal maleate). Both solid alkali metal maleate as well as dissolved asenapine in the form of its base are contained in the solvent. In particular, the alkali metal maleate that is formed, is present in the product mixture surprisingly approximately completely as a solid (e.g., ≥98% by mass). The conversion of the asenapine maleate to asenapine in the form of its free base takes place in that the maleic acid is deprotonated by the alkali metal silicate, wherein solid alkali metal maleate and dissolved asenapine in the form of its free base are formed. Moreover, free silicic acid is formed by protonation of the silicate anion. Without wishing to be bound by theory, it is assumed that the free silicic acid reacts further with elimination of water to form silicon dioxide, which is present in the product mixture as a solid (e.g., a solids fraction of about 100% by mass, based on the total mass of silicon dioxide).

In this manner, surprisingly, by the reaction of asenapine maleate with an alkali metal silicate, high conversions to asenapine in the form of its free base in high purity can be obtained. Without wishing to be bound by theory, it is believed that the driving force for the high conversions is the formation of solid silicon dioxide as well as solid alkali metal maleate, and that the high purities are achieved because there are no further reactions or side reactions of dissolved asenapine in the form of its free base.

For example, the reaction can be carried out under a protective gas, in particular comprising argon and/or nitrogen. For this purpose, a protective gas atmosphere, in particular comprising argon and/or nitrogen, can be generated by flushing gas through a reaction vessel while the reaction mixture is being prepared.

Therefore, the process according to the invention for the preparation of asenapine in the form of its free base comprises the steps of:
1) providing a reaction mixture comprising asenapine maleate and an alkali metal silicate in a solvent, wherein the reaction mixture contains the alkali metal silicate in dispersed form; and
2) reacting asenapine maleate with the alkali metal silicate in the reaction mixture provided in step 1) in order to obtain a product mixture which contains dissolved asenapine in the form of its free base and alkali metal maleate in dispersed form.

In certain embodiments, the process according to the invention comprises a third step of isolating a solution containing asenapine in the form of its free base from the product mixture obtained in step 2). Here, in particular, a solid contained in the product mixture, which solid may contain silicon dioxide and alkali metal maleate, for example, is removed from the product mixture in order to obtain a solution containing asenapine in the form of its free base. In particular, the isolation can be carried out by means of a filtration, a solid phase extraction, a sedimentation, a decanting, or by means of a centrifugation. Said isolation can simplify further processing of the asenapine in the form of its free base. Otherwise, the product mixture contains solids, such as silicon dioxide and alkali metal maleate, which would be present as impurities in particular during the preparation of a matrix layer and thus could affect the quality of the matrix layer to be prepared. In particular, the solution obtained can be used directly for the preparation of a matrix layer.

According to certain embodiments the solvent used in step 1) contains water. For example, water can be used to increase the solubility of the asenapine maleate and/or the solubility of the alkali metal silicate, as a result of which the conversion takes place faster. In particular, the solvent used in step 1) contains water and asenapine maleate in a molar ratio of 4 or less parts of water, preferably 3 or less parts of water, more preferably from 3 to ¼ parts of water, and most preferably from 2 to ⅓ parts of water, each based on 1 part of asenapine maleate. A molar ratio between water and asenapine maleate in the ranges defined above leads to a high conversion of asenapine maleate to asenapine in the form of its free base and a high purity of asenapine in the form of its free base. This is surprising and unexpected, since in general, without wishing to be bound by theory, it can be assumed that one of the driving forces of the reaction, namely the formation of silicon dioxide, which takes place with elimination of water, should be inhibited due to the presence of additional water in the reaction mixture. Accordingly, on the contrary, a lower conversion would have to be expected due to the addition of water. Surprisingly, it was also found that a molar ratio of more than 4 parts of water based on 1 part of asenapine maleate significantly slowed down the conversion of the asenapine maleate.

In certain embodiments, the alkali metal silicate is selected from the group consisting of sodium metasilicate, sodium trisilicate, potassium silicate and mixtures thereof. Sodium metasilicate is particularly preferred. Furthermore, the alkali metal silicate can also contain heterogeneous alkali metal metasilicates which, in addition to alkali metal cations, also can contain alkaline earth metal cations, such as, for example, calcium and/or magnesium, or earth metal cations such as aluminum. Examples are potassium aluminum disilicates or sodium aluminosilicates, for example AlNa₁₂SiO₅

According to certain embodiments, the d50 particle diameter of the alkali metal silicate used in step 1) is 125 μm or more, or less than 125 μm. Alternatively, the d80 particle diameter can be less than 200 μm. Surprisingly, it has here been found that particularly high conversions in a shortened reaction time, for example within one day, could be achieved by using alkali metal silicates with a d50 particle diameter of less than 125 μm or a d80 particle diameter of less than 200 μm.

In certain embodiments, the solvent used in step 1) contains alkali metal silicate and asenapine maleate in a molar ratio of 1 or more parts of alkali metal silicate, from 1 to 10 parts alkali metal silicate, from 1 to 5 parts alkali metal silicate, from 1 to 4 parts alkali metal silicate, from 1 to 3 parts of alkali metal silicate, or from 1 to 2 parts of alkali metal silicate, in each case based on 1 part of asenapine maleate. It was found, surprisingly, that its increased molar ratio of the alkali metal silicate based on the asenapine maleate leads to improved conversions in shorter periods of time, for example within one day.

According to certain embodiments, the solvent in step 1) contains an alcoholic solvent. The alcoholic solvent is preferably selected from the group consisting of methanol, ethanol, 1-propanol, 2-propanol and mixtures thereof. Ethanol is particularly preferred as an alcoholic solvent. In particular, a mass fraction of the alcoholic solvent based on the total mass of the reaction mixture provided in step 1) is 40% by mass to 97% by mass, preferably 45% by mass to 70% by mass.

In certain embodiments, the reaction of the process is carried out at a temperature of 15° C. to 45° C., at a temperature of 15° C. to 25° C., or at a temperature of 35° C. to 45° C. In particular, in a temperature range from 35° C. to 45° C., a high conversion can be achieved in a shortened time.

In certain embodiments, the mass fraction of asenapine maleate is 1% by mass to 40% by mass, preferably 2% by mass to 35% by mass, more preferably 15% by mass to 30% by mass, based on the total mass of the reaction mixture provided in step 1).

In certain embodiments, step 2) is carried out for a period of 2 h to 144 h, preferably for a period of 8 h to 60 h, particularly preferably for a period of 12 h to 50 h.

According to certain embodiments, the conversion in step 2) is 80% or more, preferably 90% or more, particularly preferably 99% or more, wherein the conversion is determined by means of HPLC as indicated above as well as in the examples and below. It is thus clear that very high conversions of asenapine maleate to form asenapine in the form of its free base can be achieved with the process according to the invention.

According to certain embodiments, asenapine in the form of its free base having a purity of 99.0% or more, preferably 99.5% or more, particularly preferably 99.95% or more is formed in step 2), wherein the purity is determined by means of HPLC as indicated above. This makes it clear that asenapine in the form of its free base can be obtained with a high degree of purity.

According to certain embodiments, antioxidants are used in step 1) as part of the reaction mixture, wherein the antioxidants are selected in particular from the group consisting of α-tocopherol, sodium metabisulfite, which is preferably used as an aqueous solution, in particular as a 10% to 40% solution, in step 1), ascorbyl palmitate, which is preferably used as an ethanolic solution, in particular as a 5% to 15% ethanolic solution, in step 1), and mixtures thereof. Using antioxidants, the purity of asenapine in the form of its free base can be increased significantly, as the antioxidants can reduce the formation of degradation products. In particular, the mass fraction of α-tocopherol is 0.01% by mass to 0.5% by mass, based on the total mass of the reaction mixture provided in step 1). Additionally or alternatively, the mass fraction of sodium metabisulfite can be 0.01% by mass to 0.5% by mass, based on the total mass of the reaction mixture provided in step 1). Additionally or alternatively, the mass fraction of ascorbyl palmitate can be 0.05% by mass to 1.0% by mass, based on the total mass of the reaction mixture provided in step 1).

### Process for the Preparation of an Active Ingredient-Containing Layer for Use in a TTS

According to a second aspect, the present invention relates to a process for the preparation of an active ingredient-containing layer, in particular an active ingredient-containing matrix layer, for use in a transdermal therapeutic system, wherein the process comprises the following steps of:
i) preparing asenapine in the form of its free base by means of the process according to the first aspect of the invention;
ii) combining at least the asenapine in the form of its free base obtained in step i) and a polymer in a further solvent in order to obtain a coating composition, wherein the asenapine in the form of its free base obtained in step i) and used in step ii) is preferably contained in the solvent used in step ii), and is more preferably present in a solution isolated according to step 3) according certain embodiments from the first aspect of the invention;
iii) coating the coating composition on a back layer, a peelable film or an intermediate film; and
iv) drying the coated coating composition to form the active ingredient-containing layer.

In particular, an active ingredient-containing layer can be prepared in a simple manner by this process because the asenapine in the form of its free base, which is prepared according to step i), is already present in a sufficient amount by the high conversion as well as in sufficiently high purity and no further purification is required.

In particular, the active ingredient-containing layer formed on the back layer, on the peelable film, or on the intermediate film after drying iv) is a matrix layer.

In particular, the asenapine maleate can be used in a solution obtained according to step 2) of the process according to the first aspect of the invention, so that only a few process steps in total are necessary to obtain an active ingredient-containing layer containing asenapine in the form of its free base.

The second aspect of the present invention, accordingly, has the technical features as well as the effects and advantages as the embodiments of the first aspect of the invention.

According to certain embodiments, the solvent of step ii) is selected from alcoholic solvents, especially methanol, ethanol, isopropanol and mixtures thereof, and from non-alcoholic solvents, in particular ethyl acetate, hexane, n-heptane, petroleum ether, toluene, and mixtures thereof. The solvent is particularly preferably selected from ethanol and ethyl acetate.

The polymer used in step ii) ensures a sufficient cohesion of the matrix layer. According to certain embodiments, the polymer can also ensure sufficient adhesion. In such embodiments, the polymer is selected from pressure-sensitive adhesive polymers.

In certain embodiments, the polymer is an acrylic polymer and preferably a copolymer based on vinyl acetate, 2-ethylhexyl acrylate, 2-hydroxyethyl acrylate and glycidyl methacrylate, or based on vinyl acetate, 2-ethylhexyl acrylate, and 2-hydroxyethyl acrylate, which is provided as a solution, and is preferably present as a solution in ethyl acetate, n-heptane, methanol, ethanol and mixtures thereof with a solids content of 30% by mass to 60% by mass.

According to certain embodiments, antioxidants such as α-tocopherol, sodium metabisulfite, which is preferably provided in an aqueous solution which is in particular a 10% to 40% aqueous solution, ascorbyl palmitate, which is preferably provided in an ethanolic solution which is in particular a 5% to 15% ethanolic solution; a triglyceride and/or a polyvinylpyrrolidone are combined with the isolated solution containing asenapine in the form of its free base and the acrylic polymer in the solvent in step ii) to obtain the coating composition.

In particular, the triglyceride used is a medium chain triglyceride.

According to certain embodiments, the active ingredient-containing layer formed in step iv) comprises
A) 4% by mass to 12% by mass asenapine in the form of its free base; and
B) 65% by mass to 85% by mass acrylic polymer;
C) 5% by mass to 15% by mass polyvinylpyrrolidone;
D) 5% by mass to 15% by mass triglyceride;
E) 0.1% by mass to 0.5% by mass ascorbyl palmitate;
F) 0.05% by mass to 0.3% by mass sodium metabisulfite;
G) 0.01% by mass to 0.1% by mass α-tocopherol; and
H) optionally 0.1 to 1% by mass aluminum acetylacetonate, based on the total mass of the active ingredient-containing layer obtained in step iv) from the coating composition. Aluminum acetylacetonate can be used in particular when the coating composition contains a crosslinking agent.

According to certain embodiments, the drying in step iv) is carried out at a temperature of 50° C. to 90° C., particularly preferably at a temperature of 60° C. to 85° C.

According to certain embodiments, the active ingredient-containing layer formed has a grammage in a range from 50 to 90 g/m² or 90 to 230 g/m², preferably from 110 to 210 g/m², and most preferably from 120 to 170 g/m².

According to the invention, the active ingredient-containing layer, which is in particular a matrix layer, contains asenapine in the form of its free base in a therapeutically effective amount.

In certain embodiments, the active ingredient-containing layer is a matrix layer.

The asenapine in the matrix layer may be completely dissolved, or the matrix layer composition may contain asenapine particles consisting of the free base of asenapine.

Without wishing to be bound by theory, it is believed that the amount of asenapine in the form of its free base is important for a good release of the active ingredient, and can be adjusted, for example, by the asenapine concentration. Thus, in certain embodiments, the amount of asenapine in the matrix layer composition ranges from 2 to 20%, preferably from 3 to 15%, and more preferably from 4 to 12% of the matrix layer composition.

According to certain embodiments, the asenapine in the form of its free base in the active ingredient-containing layer formed has a purity of 95.0% or more, preferably 99.0% or more, particularly preferably 99.95% or more, wherein the purity is determined by means of quantitative HPLC. Quantitative HPLC can be carried out using reverse phase HPLC with UV detection. In particular, the following conditions can be used if HPLC is carried out isocratically:
Column: Octadecyl phase according to Ph. Eur. 2.2.29 (USP phase L1) Kromasil C18 125 mm×4.0 mm; 5 μm or equivalent
Mobile phase: KH₂PO₄/methanol/TEA (45:55:0.1; v:v:v); pH 2.5±0.05 (TEA=triethylamine)
Gradient: isocratic
Flow: 1.0 ml
Injection volume: 30 μl
Column temperature: 40° C.
Wavelength: 225 nm, 270 nm and 3-D field; evaluation is carried out at 270 nm
Run time: 10 min
   Furthermore, the following conditions can be used when HPLC is carried out with a gradient:
Column: Octadecyl phase according to Ph. Eur. 2.2.29 (USP phase L1) Kinetex C18 EVO 100 mm×4.6 mm; 2.1 μm or equivalent
Mobile phase: A: 0.02 mol KH₂PO₄ buffer/methanol/TEA (70:30:0.1; v:v:v) adjusted to pH 2.5 B: 0.02 mol KH₂PO₄ buffer/methanol/TEA (30:70:0.1; v:v:v); adjusted to pH 2.5 (TEA=triethylamine)
Flow: 1.0 ml
Injection volume: 30 μl
Column temperature: 40° C.
Wavelength: 225 nm, 270 nm and 3-D field; evaluation is carried out at 225 nm
Run time: 32 min
Gradient profile: 0.00 min: A: 100% B: 0% 12.00 min: A: 40% B: 60% 18.00 min: A: 0% B: 100% 27.00 min: A: 0% B: 100% 27.01 min: A: 100% B: 0% 32.00 min: A: 100% B: 0%

According to certain embodiments, drying in step iv) is carried out for a period of 10 min to 60 min.

In certain embodiments, a mass fraction of asenapine in the form of its free base is 95% by mass or more, preferably 99% by mass or more, particularly preferably 100% by mass, based on the total mass of asenapine in the active ingredient-containing layer formed.

According to certain embodiments, drying in step iv) is carried out in two stages, wherein drying in the first stage is carried out at 15° C. to 25° C. for a period of 5 min to 15 min, and subsequently drying in the second stage is carried out at 60° C. to 85° C. for a period of 10 min to 40 min.

### Asenapine in the Form of its Free Base

According to a third aspect, the present invention relates to asenapine in the form of its free base, obtainable by a process according to the first aspect of the invention. Asenapine in the form of its free base has in particular a purity of 99.0% or more, preferably 99.5% or more, particularly preferably 99.95% or more, wherein the purity is determined by means of HPLC, as indicated above, wherein the asenapine in the form of its free base is preferably present in a solvent and particularly preferably in a solution isolated according to step 3) according to the first aspect of the invention.

The third aspect of the invention, accordingly, has the technical features as well as the effects and advantages as the embodiments of the first aspect of the invention.

Asenapine in the form of its free base in high purity may be obtained directly from the preparation process without the need for further costly and complex purification steps.

### Active Ingredient-Containing Layer

According to a fourth aspect, the present invention relates to an active ingredient-containing layer which, in particular, is a matrix layer, for use in a transdermal therapeutic system, obtainable by a process according to the second aspect of the invention, wherein the asenapine in the form of its free base in the active ingredient-containing layer preferably has a purity of 99.0% or more, preferably 99.5% or more, particularly preferably 99.95% or more, wherein the purity is determined by means of HPLC, as indicated above.

The fourth aspect of the invention, accordingly, has the technical features as well as the effects and advantages as the embodiments of the second aspect of the invention.

Thus, an active ingredient-containing layer which, in particular, is a matrix layer, can be provided in high purity without additional purification steps.

The active ingredient-containing layer comprises a therapeutically effective amount of asenapine in the form of its free base.

### TTS Containing an Active Ingredient-Containing Layer

According to a fifth aspect, the present invention relates to a transdermal therapeutic system containing an active ingredient-containing layer according to the fourth aspect of the invention, which is obtainable by the process according to the second aspect of the invention.

The fifth aspect of the invention, accordingly, has the technical features as well as the effects and advantages as the embodiments of the second and the fourth aspect of the invention. Thus, within the scope of the present invention, a TTS can be provided which contains asenapine in the form of its free base, as a result of which said TTS has improved skin permeation properties, for example compared to a TTS containing asenapine maleate. Furthermore, solids, in particular, which arise during the preparation of asenapine in the form of its free base, can be easily removed so that they can be excluded as impurities in the TTS prepared.

In particular, the transdermal therapeutic system has an asenapine-containing (in the form of its free base) self-adhesive layer structure.

The transdermal therapeutic system according to the present invention is preferably a transdermal therapeutic system for the transdermal administration of asenapine in the form of its free base, wherein the TTS in particular is an asenapine-containing (in the form of its free base) self-adhesive layer structure, comprising:
A) a back layer;
B) an asenapine-containing (in the form of its free base) active ingredient-containing layer, which is in particular a matrix layer, consisting of a composition comprising:
   1. asenapine in the form of its free base; and
   2. a polymer selected from acrylic polymers;
      wherein the transdermal therapeutic system has a release area of 5 to 100 cm².

In particular, the back layer is essentially asenapine-impermeable.

The TTS according to the present invention may be a TTS of the matrix type or a TTS of the reservoir type, and is preferably a TTS of the matrix type.

The TTS can also contain components known to the person skilled in the art, such as, for example, a skin contact layer and/or a top layer. When the TTS is of the reservoir type, the TTS may contain other components known to the person skilled in the art, such as a rate controlling membrane.

### EXAMPLES

The present invention will now be described more fully with reference to the accompanying examples. It should be understood, however, that the following description is illustrative only and should in no way be taken as a limitation on the invention. The numerical values given in the examples with regard to the amount of the ingredients in the composition/in the reaction mixture or the grammage may vary slightly due to preparation fluctuations.

### Examples 1a-c

### Reaction Mixtures

The masses and the fractions of the ingredients of the reaction mixtures, which were used in Examples 1a-c, are summarized in Table 1.1. All percentages in Table 1.1 refer to mass percent (% by mass).

TABLE 1.1

| | Ex. 1a | | Ex. 1b | | Ex. 1c | |
|---|---|---|---|---|---|---|
| | Mass | Fraction | Mass | Fraction | Mass | Fraction |
| Ingredient | [g] | [%] | [g] | [%] | [g] | [%] |
| Asenapine maleate | 0.6627 | 19.33 | 0.6772 | 24.29 | 0.6794 | 24.29 |
| Water (total)* | 0.0987 | 2.88 | 0.0319 | 1.14 | 0.0320 | 1.14 |
| Sodium metasilicate | 0.3966 | 11.57 | 0.4104 | 14.72 | 0.4118 | 14.72 |
| Ethanol with 1% | 2.2527 | 65.70 | 1.6499 | 59.18 | 1.6553 | 59.18 |
| MEK (total)** | | | | | | |
| α-Tocopherol | 0.0024 | 0.07 | 0.0025 | 0.09 | 0.0025 | 0.09 |
| Ascorbyl palmitate | 0.0095 | 0.28 | 0.0097 | 0.35 | 0.0097 | 0.35 |
| solution [10% in ethanol | | | | | | |
| with 1% MEK]*** | | | | | | |
| Sodium metabisulfite | 0.0060 | 0.18 | 0.0061 | 0.22 | 0.0062 | 0.22 |
| solution [30% in | | | | | | |
| water]**** | | | | | | |

| | | | |
|---|---|---|---|
| Temperature [C.] | 20.0 | 40.0 | 20.0 |

*Sum of the masses/fractions of the water, which is added in the form of a 30% sodium metabisulfite solution, and the remaining water, which is added as part of the solvent of the reaction mixture.
**Sum of the masses/fractions of the ethanol, which is present in the form of 10% ascorbyl palmitate solution, and the remaining ethanol, which is added as part of the solvent of the reaction mixture.
***Mass and fraction refer to ascorbyl palmitate.
****Mass and fraction refer to sodium metabisulfite.

### Preparation of Asenapine in the Form of its Free Base

For the preparation of the reaction mixture for Examples 1a-c, an asenapine maleate base mixture was prepared first. For this purpose, a glass vessel was charged with α-tocopherol (0.0536 g). Thereafter, ethanol with 1% MEK (33.6898 g) as the solvent was added and the resulting mixture was stirred at 300 rpm. Then, a 30% aqueous sodium metabisulfite solution (0.4419 g) and a 10% ethanolic ascorbyl palmitate solution (2.0916 g) were added in succession, each with stirring at 300 rpm. Subsequently asenapine maleate was added (14.6009 g) and the asenapine base mixture was stirred at 300 rpm.

For providing the reaction mixture of Example la, 2.3090 g of this asenapine maleate base mixture were taken out of the glass vessel with stirring and placed in a reaction vessel, whereupon additional ethanol with 1% MEK (0.6381 g) as solvent was added and the resulting mixture was stirred at 900 rpm. In addition, water (see Table 1.1 for the mass) was added as a further solvent with stirring at 900 rpm. Then, ground sodium metasilicate (see Table 1.1 for the mass) was added in order to obtain the reaction mixture according to Example 1a (see Table 1.1). Then, the reaction vessel was flushed with argon and the reaction mixture was stirred for approximately 3 days at 20° C. and at 300 rpm for the conversion of asenapine maleate to asenapine in the form of its free base.

For providing the reaction mixture of Example 1c, 44.803 g asenapine maleate base mixture were taken out with stirring and placed in a glass vessel, whereupon water (0.3324 g) was added to the asenapine maleate base mixture with stirring in order to obtain a precursor mixture. Then, the resulting precursor mixture (21.4939 g) was taken out with stirring and placed in a further glass vessel. Thereafter, ground sodium metasilicate (3.7108 g) was added. Then, the glass vessel was flushed with argon and stirred at 300 rpm. Thereafter, 2.7877 g of the mixture thus obtained were taken out with stirring at 500 rpm in order to obtain the reaction mixture according to Example 1b (see Table 1.1). This reaction mixture was placed in a reaction vessel and stirred for 3 days at 40° C. and 300 rpm for the conversion of asenapine maleate to asenapine in the form of its free base.

For providing the reaction mixture of Example 1c, 44.803 g of asenapine maleate base mixture were taken out with stirring and placed in a glass vessel, whereupon water (0,3324 g) was added to the asenapine maleate base mixture with stirring in order to obtain a precursor mixture. Then, the resulting precursor mixture (21.4939 g) was taken out with stirring and placed in another glass vessel. Thereafter, ground sodium metasilicate (3.7108 g) was added. Then, the glass vessel was flushed with argon and stirred at 300 rpm. Thereafter, 2.7968 g of the mixture thus obtained were taken out with stirring at 500 rpm in order to obtain the reaction mixture according to Example 1c (see Table 1.1). This reaction mixture was placed in a reaction vessel and stirred for 3 days at 20° C. and 300 rpm for the conversion of asenapine maleate to asenapine in the form of its free base.

### Conversion of Asenapine Maleate and Purity of Asenapine in the Form of its Free Base

For reaction analysis, a liquid sample of 500 μl were taken out of the product mixtures of Examples 1 a-c in each case after one day, after two days and after three days of reaction by means of an Eppendorf pipette. After the first and second sampling, the reaction vessel in each case was flushed again with argon and stirring was continued.

For determining the conversion, a total mass of asenapine (which originates from asenapine in the form of its free base as well as asenapine maleate) and a mass of maleic acid of the liquid samples were determined by means of quantitative HPLC (see below in Examples 5a-d). In this way, the mass fraction of asenapine in the form of its free base can be determined by calculation. The conversion of asenapine maleate (see Table 1.2) in this case corresponds to the mass fraction of asenapine in the form of its free base, based on the total mass of asenapine of the sample.

The liquid samples were also used to determine any degradation products of asenapine by means of quantitative HPLC. The smaller the sum of the degradation products, the higher the purity of asenapine in the form of its free base.

The values of conversions of asenapine maleate as well as the sums of the degradation products are summarized in Table 1.2.

TABLE 1.2

| | Conversion of | | | Sum of the | | |
|---|---|---|---|---|---|---|
| | asenapine maleate | | | degradation products | | |
| Ex. | Day 1 | Day 2 | Day 3 | Day 1 | Day 2 | Day 3 |
| Ex. 1a | 79.84% | 83.77% | 87.68% | 0.04% | 0.01% | 0.01% |
| Ex. 1b | 97.85% | 99.36% | 99.75% | 0.00% | 0.05% | 0.05% |
| Ex. 1c | 84.66% | 97.53% | 99.51% | 0.01% | 0.01% | 0.01% |

From Table 1.2 it can be seen that high conversions and high purities could be obtained with the reaction mixtures of Examples 1a-c. It was also found, surprisingly, that a lower fraction of water (cf. Ex. 1a and Ex. 1c) and a higher reaction temperature of 40° C. (cf. Ex. 1a and Ex. 1b) result in a faster conversion, i.e. a higher conversion is achieved within a shorter time, from asenapine maleate to asenapine in the form of its free base.

### Reference Examples 1a-c

### Reaction Mixtures

The masses as well as the fractions of the ingredients of the reaction mixtures, which were used in Reference Examples 1a-c, are summarized in Table 2.1. All percentages in Table 2.1 refer to mass percent (% by mass).

TABLE 2.1

| | Ref. Ex. 1a | | Ref. Ex. 1b | | Ref. Ex. 1c | |
|---|---|---|---|---|---|---|
| | Mass | Fraction | Mass | Fraction | Mass | Fraction |
| Ingredient | [g] | [%] | [g] | [%] | [g] | [%] |
| Asenapine maleate | 0.7060 | 24.73 | 0.6860 | 24.46 | 0.6697 | 24.20 |
| Water (total)* | 0.0332 | 1.16 | 0.0323 | 1.15 | 0.0315 | 1.14 |
| Aluminum silicate | 0.3767 | 13.19 | — | — | — | — |
| Calcium silicate, meta | — | — | 0.3964 | 14.13 | — | — |
| Magnesium silicate | — | — | — | — | 0.4163 | 15.04 |
| monohydrate | | | | | | |
| Ethanol with 1% | 1.7201 | 60.25 | 1.6713 | 59.59 | 1.6315 | 58.96 |
| MEK (total)** | | | | | | |
| α-Tocopherol | 0.0026 | 0.09 | 0.0025 | 0.09 | 0.0025 | 0.09 |
| Ascorbyl palmitate | 0.0101 | 0.35 | 0.0098 | 0.35 | 0.0096 | 0.35 |
| solution [10% by mass in ethanol | | | | | | |
| with 1% MEK]*** | | | | | | |
| Sodium metabisulfite | 0.0064 | 0.22 | 0.0062 | 0.22 | 0.0061 | 0.22 |
| solution [30% by | | | | | | |
| mass in water]**** | | | | | | |

*Sum of the masses/fractions of the water, which is added in the form of a 30% sodium metabisulfite solution, and the remaining water, which is added as part of the solvent of the reaction mixture.
**Sum of the masses/fractions of the ethanol, which is present in the form of 10% ascorbyl palmitate solution, and the remaining ethanol, which is added as part of the solvent of the reaction mixture.
***Mass and fraction refer to ascorbyl palmitate.
****Mass and fraction refer to sodium metabisulfite.

### Preparation of Asenapine in the Form of its Free Base

For providing the reaction mixtures of Reference Example 1a-c, the precursor mixture of Example 1c was used first.

For providing the reaction mixture of Reference Example 1a, the precursor mixture (2.4784/g was taken out with stirring at 650 rpm and placed in a reaction vessel. Aluminum silicate (0.3767 g) was added in order to obtain the reaction mixture according to Reference Example 1a. Then, the reaction vessel was flushed with argon. The reaction mixture was stirred at 300 rpm and 20.0° C. for 3 days for the conversion of asenapine maleate to asenapine in the form of its free base.

For providing the reaction mixture of Reference Example 1b, the precursor mixture (2.4082 g) was taken out with stirring at 650 rpm and placed in a reaction vessel. Calcium silicate, meta (0.3964 g) was added in order to obtain the reaction mixture according to Reference Example 1b. Then, the reaction vessel was flushed with argon. The reaction mixture was stirred at 300 rpm and 20.0° C. for 3 days for the conversion of asenapine maleate to asenapine in the form of its free base.

For providing the reaction mixture of Reference Example 1c, the precursor mixture (2.3508 g) was taken out with stirring at 455 rpm and placed in a reaction vessel. Magnesium silicate monohydrate (0.4163 g) was added to this precursor mixture in order to obtain the reaction mixture according to Reference Example 1c. Thereafter, the reaction vessel was flushed with argon and the reaction mixture was stirred at 300 rpm and 20.0° C. for 3 days for the conversion of asenapine maleate to asenapine in the form of its free base.

### Conversion of Asenapine Maleate and Purity of Asenapine in the Form of its Free Base

The conversion of asenapine maleate and the sum of the degradation products were determined analogously to Examples 1a-c. The conversions as well as the sum of the degradation products are summarized in Table 2.2.

TABLE 2.2

| | Conversion of | | | Sum of the | | |
|---|---|---|---|---|---|---|
| | asenapine maleate | | | degradation products | | |
| Ex. | Day 1 | Day 2 | Day 3 | Day 1 | Day 2 | Day 3 |
| Ref. Ex. 1a | — | — | — | 0.02% | 0.02% | 0.04% |
| Ref. Ex. 1b | — | — | — | 0.03% | 0.02% | 0.05% |
| Ref. Ex. 1c | — | — | — | 0.02% | 0.02% | 0.02% |

Surprisingly, it was found that silicates with higher-valent cations such as Ca²⁺, Mg²⁺ and Al³⁺ (see Ref. Ex. 1a-c in Table 2.2), in contrast to silicates with sodium cations (cf. Example 1a-c in Table 1.2), do not result in any conversion.

### Example 2a and Reference Examples 2a-c

### Reaction Mixtures

The masses as well as the fractions of the ingredients of the reaction mixtures, which were used in Example 2a as well as in Reference Examples 2a-c, are summarized in Table 3.1. All percentages in Table 3.1 refer to mass percent (% by mass).

TABLE 3.1

| | Ex. 2a | | Ref. Ex. 2a | | Ref. Ex. 2b | | Ref. Ex. 2c | |
|---|---|---|---|---|---|---|---|---|
| | Mass | Fraction | Mass | Fraction | Mass | Fraction | Mass | Fraction |
| Ingredient | [g] | [%] | [g] | [%] | [g] | [%] | [g] | [%] |
| Asenapine maleate | 0.5809 | 21.17 | 0.6208 | 21.84 | 0.5961 | 21.45 | 0.58956 | 21.01 |
| Water (total)* | 0.0273 | 1.00 | 0.0292 | 1.03 | 0.0280 | 1.01 | 0.02773 | 0.99 |
| Sodium metasilicate | 0.7042 | 25.67 | — | — | — | — | — | |
| Aluminum silicate | — | — | 0.6635 | 23.34 | — | — | — | |
| Calcium silicate, meta | — | — | — | — | 0.6870 | 24.72 | — | |
| Magnesium silicate | — | — | — | — | — | — | 0.7365 | 26.25 |
| monohydrate | | | | | | | | |
| Ethanol with 1% | 1.4152 | 51.59 | 1.5123 | 53.20 | 1.4523 | 52.25 | 1.43635 | 51.19 |
| MEK (total)** | | | | | | | | |
| α-tocopherol | 0.0021 | 0.08 | 0.0023 | 0.08 | 0.0022 | 0.08 | 0.00216 | 0.08 |
| Ascorbyl palmitate | 0.0083 | 0.30 | 0.0089 | 0.31 | 0.0085 | 0.31 | 0.00845 | 0.30 |
| solution [10% in ethanol | | | | | | | | |
| with 1% MEK]*** | | | | | | | | |
| Sodium metabisulfite | 0.0053 | 0.19 | 0.0056 | 0.20 | 0.0054 | 0.19 | 0.00536 | 0.19 |
| solution [30% in | | | | | | | | |
| water]**** | | | | | | | | |

*Sum of the masses/fractions of the water, which is added in the form of a 30% sodium metabisulfite solution, and the remaining water, which is added as part of the solvent of the reaction mixture.
**Sum of the masses/fractions of the ethanol, which is present in the form of 10% ascorbyl palmitate solution, and the remaining ethanol, which is added as part of the solvent of the reaction mixture.
***Mass and fraction refer to ascorbyl palmitate.
****Mass and fraction refer to sodium metabisulfite.

### Preparation of Asenapine in the Form of its Free Base

For providing the reaction mixture of Example 2a and Reference Examples 2a-d, the precursor mixture according to Example 1c was used.

For Example 2a, 2.0391 g of the precursor mixture were taken out with stirring at 650 rpm and placed in a reaction vessel. Thereafter, sodium metasilicate (0.7042 g) was added in order to obtain the reaction mixture according to Example 2a (see Table 3.1). Thereafter, the reaction vessel was flushed with argon, whereupon the reaction mixture was stirred at 300 rpm and 20.0° C. for 3 days for the conversion of asenapine maleate to asenapine in the form of its free base.

For Reference Example 2a, 2.1791 g of the precursor mixture were taken out with stirring at 650 rpm and placed in a reaction vessel. Thereafter, aluminum silicate (0.6635 g) was added in order to obtain the reaction mixture according to Reference Example 2a (see Table 3.1). Thereafter, the reaction vessel was flushed with argon, whereupon the reaction mixture was stirred at 300 rpm and 20.0° C. for 3 days for the conversion of asenapine maleate to asenapine in the form of its free base.

For Reference Example 2b, 2.0926 g of the precursor mixture (from Example 1c) were taken out with stirring at 650 rpm and placed in a reaction vessel. Thereafter, calcium silicate, meta (0.6870 g) was added in order to obtain the reaction mixture according to Reference Example 2b (see Table 3.1). Thereafter, the reaction vessel was flushed with argon, whereupon the reaction mixture was stirred at 300 rpm and 20.0° C. for 3 days for the conversion of asenapine maleate to asenapine in the form of its free base.

For Reference Example 2c, 2.0696 g of the precursor mixture were taken out with stirring at 455 rpm and placed in a reaction vessel. Thereafter, magnesium silicate monohydrate (0.7365 g) was added in order to obtain the reaction mixture according to Reference Example 2c (see Table 3.1). Thereafter, the reaction vessel was flushed with argon, whereupon the reaction mixture was stirred at 300 rpm and 20.0° C. for 3 days for the conversion of asenapine maleate to asenapine in the form of its free base.

### Conversion of Asenapine Maleate and Purity of Asenapine in the Form of its Free Base

The conversion of asenapine maleate and the purity of asenapine in the form of its free base were determined analogously to Examples 1a-c. The conversions as well as the sum of the degradation products are summarized in Table 3.2.

TABLE 3.2

| | Conversion of | | | Sum of the | | |
|---|---|---|---|---|---|---|
| | asenapine maleate | | | degradation products | | |
| Ex. | Day 1 | Day 2 | Day 3 | Day 1 | Day 2 | Day 3 |
| Ex. 2a | 97.35% | 99.67% | 99.66 | 0.01 | 0.01 | 0.01% |
| Ref. Ex. 2a | — | — | — | 0.02% | 0.03% | 0.05% |
| Ref. Ex. 2b | — | — | — | 0.03% | 0.03% | 0.05% |
| Ref. Ex. 2c | — | — | — | 0.02% | 0.02% | 0.04% |

From Table 3.2 it can be seen that the use of a higher mass fraction of sodium metasilicate resulted in a higher and faster conversion, e.g., achievement of a conversion in a shorter time, at high purity (see Ex. 2a), wherein no conversion took place when using calcium silicate, magnesium silicate and aluminum silicate in higher parts by mass (see Ref. Ex. 2a-c).

### Examples 3a and 3b

### Reaction Mixtures

The masses as well as the fractions of the ingredients of the reaction mixtures, which were used in Examples 3a and 3b, are summarized in Table 4.1. All percentages in Table 4.1 refer to mass percent (% by mass).

TABLE 4.1

| | Ex. 3a | | Ex. 3b | |
|---|---|---|---|---|
| | Mass | Fraction | Mass | Fraction |
| Ingredient | [g] | [%] | [g] | [%] |
| Asenapine maleate | 0.6852 | 24.78 | 0.6076 | 21.55 |
| Water (total) * | 0.0309 | 1.12 | 0.0274 | 0.97 |
| Sodium metasilicate | 0.4150 | 15.01 | 0.7355 | 26.09 |
| Ethanol with 1% MEK (total) ** | 1.6248 | 58.76 | 1.4407 | 51.10 |
| α-Tocopherol | 0.0015 | 0.05 | 0.0013 | 0.05 |
| Ascorbyl palmitate solution | 0.0051 | 0.18 | 0.0045 | 0.16 |
| [10% in ethanol with 1% | | | | |
| MEK] *** | | | | |
| Sodium metabisulfite solution | 0.0026 | 0.09 | 0.0023 | 0.08 |
| [30% in water] **** | | | | |

* Sum of the masses/fractions of the water, which is added in the form of a 30% sodium metabisulfite solution, and the remaining water, which is added as part of the solvent of the reaction mixture.
** Sum of the masses/fractions of the ethanol, which is present in the form of 10% ascorbyl palmitate solution, and the remaining ethanol, which is added as part of the solvent of the reaction mixture.
*** Mass and fraction refer to ascorbyl palmitate.
**** Mass and fraction refer to sodium metabisulfite.

### Preparation of Asenapine in the Form of its Free Base

For the preparation of the reaction mixture of Examples 3a and 3b first, a base mixture of asenapine maleate prepared first. For this purpose, α-tocopherol (0.0146 g) was weighed out in a glass vessel. Thereafter, ethanol with 1% MEK (15.7001 g) was added as the solvent and the mixture was stirred at 300 rpm. A 30% aqueous sodium metabisulfite solution (0.0856 g) and a 10% ethanolic ascorbyl palmitate solution (0.5047 g) were then added in succession, each with stirring at 300 rpm. Next, asenapine maleate (6.8126 g) was added, whereupon the resulting mixture was further stirred at 300 rpm. Then, water (0.2471 g) as a further solvent was added with stirring in order to obtain the base mixture of asenapine.

For providing the reaction mixture of Example 3a, 2.3500 g of this base mixture of asenapine maleate were taken out with stirring at 500 rpm and placed in a reaction vessel. Ground sodium metasilicate (0.4150 g) was added in order to obtain the reaction mixture according to Example 3a. Thereafter, the reaction vessel was flushed with argon. Subsequently, the reaction mixture was stirred at 300 rpm and 20.0° C. for 3 days for the conversion of asenapine maleate to asenapine in the form of its free base.

For providing the reaction mixture of Example 3b, 2.0838 g of the base mixture of asenapine maleate were taken out with stirring at 500 rpm and placed in a reaction vessel. Ground sodium metasilicate (0.7355 g) was added in order to obtain the reaction mixture according to Example 3b. Then, the reaction vessel was flushed with argon. Thereafter, the reaction mixture was stirred at 300 rpm and 20.0° C. for 3 days for the conversion of asenapine maleate to asenapine in the form of its free base.

### Conversion of Asenapine Maleate and Purity of Asenapine in the Form of its Free Base

The conversion of asenapine maleate and the purity of asenapine in the form of its free base were determined analogously to Examples 1a-c. The conversion as well as the sum of the degradation products are summarized in Table 4.2.

TABLE 4.2

| | Conversion of | | | Sum of the | | |
|---|---|---|---|---|---|---|
| | asenapine maleate | | | degradation products | | |
| Ex. | Day 1 | Day 2 | Day 3 | Day 1 | Day 2 | Day 3 |
| Ex. 3a | 67.02% | 81.05% | 91.80% | 0.01% | 0.00% | 0.01% |
| Ex. 3b | 97.85% | 99.36% | 99.75% | 0.01% | 0.01% | 0.01% |

From Table 4.2 it can be seen that an antioxidant fraction (in the form of sodium metabisulfite, ascorbyl palmitate as well as α-tocopherol) that is reduced in comparison with the reaction mixtures of Examples 1a-c also leads to high conversions and high purities (see Ex. 3a and 3b). Furthermore, it could be shown, surprisingly, that an increase in the mass fraction of the sodium metasilicate used (see Ex. 3b, Table 4.1) leads to higher conversions and to a faster conversion.

### Examples 4a-c

### Reaction Mixtures

The masses as well as the fractions of the ingredients of the reaction mixtures, which were used in Examples 4a-c, are summarized in Table 5.1. All percentages in Table 5.1 refer to mass percent (% by mass).

TABLE 5.1

| | Ex. 4a | | Ex. 4b | | Ex. 4c | |
|---|---|---|---|---|---|---|
| | Mass | Fraction | Mass | Fraction | Mass | Fraction |
| Ingredient | [g] | [%] | [g] | [%] | [g] | [%] |
| Asenapine maleate | 0.6833 | 22.95 | 0.6854 | 21.41 | 0.6836 | 21.12 |
| Water (total)* | 0.0470 | 1.58 | 0.0471 | 1.47 | 0.0375 | 1.16 |
| Sodium metasilicate | 0.4161 | 13.98 | 0.4160 | 12.99 | 0.4169 | 12.88 |
| Ethanol with 1% | 0.1401 | 4.71 | 0.1424 | 4.45 | 2.0990 | 64.84 |
| MEK (total) ** | | | | | | |
| Methanol | 1.6608 | 55.78 | — | — | — | — |
| 1-Propanol | — | — | 1.8812 | 58.76 | — | — |
| α-Tocopherol | 0.0055 | 0.18 | 0.0032 | 0.10 | — | — |
| Ascorbyl palmitate | 0.0156 | 0.52 | 0.0158 | 0.49 | — | — |
| solution [10% in ethanol | | | | | | |
| with 1% MEK]*** | | | | | | |
| Sodium metabisulfite | 0.0088 | 0.29 | 0.0106 | 0.33 | — | — |
| solution [30% in | | | | | | |
| water]**** | | | | | | |

*Sum of the masses/fractions of the water, which is added in the form of a 30% sodium metabisulfite solution, and the remaining water, which is added as part of the solvent of the reaction mixture.
** Sum of the masses/fractions of the ethanol, which is present in the form of 10% ascorbyl palmitate solution, and the remaining ethanol, which is added as part of the solvent of the reaction mixture.
***Mass and fraction refer to ascorbyl palmitate.
****Mass and fraction refer to sodium metabisulfite.

### Preparation of Asenapine in the Form of its Free Base

For providing the reaction mixture according to Example 4a, asenapine maleate was placed in a reaction vessel. Then, α-tocopherol and methanol (as solvent) were added. Thereafter, a 30% aqueous sodium metabisulfite solution and a 10% ethanolic ascorbyl palmitate solution were added in succession with stirring at 300 rpm. Water was added as a further solvent. Furthermore, ground sodium metasilicate was added in order to obtain the reaction mixture according to Example 4a. The reaction vessel was flushed with argon and the reaction mixture according to Example 4a was stirred at 300 rpm and 20.0° C. for 3 days for the conversion of asenapine maleate to asenapine in the form of its free base.

For providing the reaction mixture according to Example 4b, α-tocopherol was placed in a reaction vessel. Then, 1-propanol was added as a solvent and the resulting mixture was stirred at 300 rpm. Thereafter, a 30% aqueous sodium metabisulfite solution and a 10% ethanolic ascorbyl palmitate solution were added in succession with stirring at 300 rpm. Then, asenapine maleate was added and the resulting mixture was stirred at 300 rpm. Furthermore, water (as a further solvent) and then ground sodium metasilicate were added in order to obtain the reaction mixture according to Example 4b. The reaction vessel was flushed with argon and the reaction mixture according to Example 4b was stirred at 300 rpm and 20.0° C. for 3 days for the conversion of asenapine maleate to asenapine in the form of its free base.

For providing the reaction mixture according to Example 4c, asenapine maleate was placed in a reaction vessel. Then, ethanol (with 1% MEK) and water, as a solvent in each case, were added in succession. Furthermore, ground sodium metasilicate was added in order to obtain the reaction mixture according to Example 4c. The reaction vessel was flushed with argon and the reaction mixture according to Example 4c was stirred at 300 rpm and 20.0° C. for 3 days for the conversion of asenapine maleate to asenapine in the form of its free base.

The masses of the ingredients used for the reaction mixtures according to Examples 4a-c are summarized in Table 5.1.

### Conversion of Asenapine Maleate and Purity of Asenapine in the Form of its Free Base

The conversion of asenapine maleate and the sum of the degradation products were determined analogously to Examples 1a-c. The conversions and the sum of the degradation products are summarized in Table 5.2.

TABLE 5.2

| | Conversion of | | | Sum of the | | |
|---|---|---|---|---|---|---|
| | asenapine maleate | | | degradation products | | |
| Ex. | Day 1 | Day 2 | Day 3 | Day 1 | Day 2 | Day 3 |
| Ex. 4a | 32.59% | 44.90% | 57.04% | 0.01% | 0.01% | 0.01% |
| Ex. 4b | 56.28% | 63.37% | 79.87% | 0.01% | 0.01% | 0.03% |
| Ex. 4c | 71.86% | 82.58% | 94.38% | 0.01% | 0.05% | 0.05% |

From Table 5.2 it can be seen that the use of various alcoholic solvents also results in a high purity of asenapine in the form of its free base (see Ex. 4a and 4b). It was found, surprisingly, that particularly high conversions took place using ethanol (cf. Ex. 4a and 4b in Table 5.2 and Ex. 1c in Table 1.2). Furthermore, it was found, surprisingly, that even without the use of antioxidants, dissolved asenapine in the form of its free base could be obtained in high purity (see Table 5.2, Example 4c).

### Examples 5a-f

### Reaction Mixtures

The masses as well as the fractions of the ingredients of the reaction mixtures, which were used in Examples 5a-d are summarized in Table 6.1. All percentages in Table 6.1 refer to mass percent (% by mass).

TABLE 6.1

| | Ex. 5a | | Ex. 5b | | Ex. 5c | | Ex. 5d | |
|---|---|---|---|---|---|---|---|---|
| | Mass | Fraction | Mass | Fraction | Mass | Fraction | Mass | Fraction |
| Ingredient | [g] | [%] | [g] | [%] | [g] | [%] | [g] | [%] |
| Asenapine maleate | 2.5377 | 24.94 | 2.5379 | 24.94 | 2.3339 | 22.84 | 2.2388 | 21.33 |
| Water (total) * | 0.1078 | 1.06 | 0.1055 | 1.04 | 0.1142 | 1.12 | 0.1083 | 1.03 |
| Sodium metasilicate | 1.5478 | 15.21 | 1.5476 | 15.21 | 2.1350 | 20.89 | 2.7314 | 26.02 |
| Ethanol with 1% | 5.9477 | 58.45 | 5.9520 | 58.48 | 5.5996 | 54.79 | 5.3886 | 51.33 |
| MEK (total)** | | | | | | | | |
| α-Tocopherol | 0.0053 | 0.05 | 0.0058 | 0.06 | 0.0061 | 0.06 | 0.0041 | 0.04 |
| Ascorbyl palmitate | 0.0181 | 0.18 | 0.0183 | 0.18 | 0.0167 | 0.16 | 0.0153 | 0.15 |
| solution [10% in ethanol | | | | | | | | |
| with 1% MEK]*** | | | | | | | | |
| Sodium metabisulfite | 0.0115 | 0.11 | 0.0109 | 0.11 | 0.0140 | 0.14 | 0.0107 | 0.10 |
| solution [30% in | | | | | | | | |
| water] **** | | | | | | | | |

* Sum of the masses/fractions of the water, which is added in the form of a 30% sodium metabisulfite solution, and the remaining water, which is added as part of the solvent of the reaction mixture.
**Sum of the masses/fractions of the ethanol, which is present in the form of 10% ascorbyl palmitate solution, and the remaining ethanol, which is added as part of the solvent of the reaction mixture.
***Mass and fraction refer to ascorbyl palmitate.
**** Mass and fraction refer to sodium metabisulfite.

### Preparation of Asenapine in the Form of its Free Base

To give the reaction mixture according to Example 5a, a reaction vessel was first charged with α-tocopherol. Then, ethanol (with 1% MEK) was added as a solvent, whereupon the resulting mixture was stirred at 300 rpm. Thereafter, a 30% aqueous sodium metabisulfite solution and a 10% ethanolic ascorbyl palmitate solution were added in succession with stirring. Thereafter, asenapine maleate was added. The resulting mixture was stirred at 300 rpm. Then, water (as a further solvent) and subsequently sodium metasilicate (d50 particle diameter<125 μm) were added with stirring in order to obtain the reaction mixture according to Example 5a. The reaction mixture was stirred at 300 rpm and 21.0° C. for 2 days for the conversion of asenapine maleate to asenapine in the form of its free base.

Examples 5b-d were carried out in an analogous manner, wherein the reaction of Example 5b was carried out at 40° C.

The masses of the ingredients of the reaction mixtures according to Examples 5a-5d are listed in Table 6.1.

### Reaction Mixture and Filtrate

The masses as well as the fractions of the ingredients of the reaction mixture and the filtrate obtained following the reaction and used in Example 5e, are summarized in Table 6.2. Table 6.2 also shows the masses as well as the fractions of the ingredients of the product filtrate obtained with the reaction mixture according to Example 5e after the reaction has ended. All percentages in Table 6.2 refer to mass percent (% by mass).

TABLE 6.2

| | Ex. | | | |
|---|---|---|---|---|
| | Ex. 5e | | Product filtrate Ex. 5e | |
| | Mass | Fraction | Mass | Fraction |
| Ingredient | [g] | [%] | [g] | [%] |
| Asenapine maleate | 2.5256 | 24.88 | 0.0251 | 0.32 |
| Asenapine in the form of its | — | — | 1.7696 | 22.50 |
| free base | | | | |
| Water (total)* | 0.1206 | 1.19 | 0.1205 | 1.53 |
| Sodium metasilicate | 1.5411 | 15.18 | — | — |
| Ethanol with 1% MEK** | 5.9258 | 58.38 | 5.9258 | 75.34 |
| α-Tocopherol | 0.0059 | 0.06 | 0.0059 | 0.08 |
| Ascorbyl palmitate solution | 0.0189 | 0.19 | 0.0189 | 0.24 |
| [10% in ethanol with 1% | | | | |
| MEK]*** | | | | |
| Sodium metabisulfite solution | 0.0119 | 0.12 | n/a***** | — |
| [30% in water]**** | | | | |

*Sum of the masses/fractions of the water, which is added in the form of a 30% sodium metabisulfite solution, and the remaining water, which is added as part of the solvent of the reaction mixture.
**Sum of the masses/fractions of the ethanol, which is present in the form of 10% ascorbyl palmitate solution, and the remaining ethanol, which is added as part of the solvent of the reaction mixture.
***Mass and fraction refer to ascorbyl palmitate.
****Mass and fraction refer to sodium metabisulfite.
*****Not measured due to the small residual amount.

### Preparation of Asenapine in the Form of its Free Base

To give the reaction mixture according to Example 5e, a reaction vessel was first charged with α-tocopherol. Then, ethanol with 1% MEK was added as a solvent, whereupon the resulting mixture was stirred at 300 rpm. Thereafter, a 30% aqueous sodium metabisulfite solution and a 10% ethanolic ascorbyl palmitate solution were added in succession with stirring. Then, asenapine maleate was added. The resulting mixture was stirred at 300 rpm. Then, water and subsequently sodium metasilicate (d50 particle diameter<125 μm) were added with stirring in order to obtain the reaction mixture according to Example 5e. The reaction vessel was flushed with argon. The reaction mixture was stirred at 300 rpm and 20.2° C. for 2 days for the conversion of asenapine maleate to asenapine in the form of its free base. The masses of the ingredients of the reaction mixture according to Example 5e are listed in Table 6.2. Following the reaction, the solid that was present in the reaction mixture, was filtered off through a suction filter on a porcelain frit with blue band filter paper in order to obtain the filtrate of Example 5e.

### Coating Composition

The formulation of the asenapine-containing (in the form of its free base) coating compositions of Example 5f is summarized in Table 6.3 below. All percentages in Table 6.3 below refer to mass percent (% by mass).

TABLE 6.3

| | Ex. 5f | |
|---|---|---|
| Ingredient | Mass [g] | Solid fraction [%] |
| Asenapine in the form of its free | 0.8315 | 7.76 |
| base as a component of the | | |
| filtrate of Ex. 5e* | | |
| DURO-TAK 87-4287 | 7.6500 | 71.36 |
| Povidone K30 red. | 1.1005 | 10.27 |
| peroxide content | | |
| α-Tocopherol | 0.0061 | 0.06 |
| Ascorbyl palmitate solution [10% | 0.0222 | 0.21 |
| in ethanol with 1% MEK] | | |
| Sodium metabisulfite solution [30% | 0.0117 | 0.11 |
| in water] | | |
| Miglyol 812 N | 1.0987 | 10.25 |
| Ethanol with 1% MEK** | 6.0631 | — |

*Solution was obtained by filtration.
**Mass of the ethanol used as a solvent for the coating composition minus the mass of the ethanol used for the 10% ascorbyl palmitate solution.

### Preparation of the Coating Composition

In example 5f, a glass vessel was charged with α-tocopherol. In the second step, the solvent (ethanol with 1% MEK) was added. The mixture was stirred at 200 rpm. Thereafter, a 30% aqueous sodium metabisulfite solution and a 10% ethanolic ascorbyl palmitate solution were added in succession with stirring. Furthermore, Miglyol 812 N and Povidone K30 (reduced peroxide content) were added in succession with stirring. The pressure sensitive acrylic adhesive (DURO-TAK 87-4287) was added and the mixture was stirred at approximately 800 rpm for approximately 3.75 hours. Then, the resulting mixture was left overnight without stirring. Then, the product filtrate (3.5950 g) obtained by virtue of Example 5e and containing asenapine in the form of its free base, was added to the mixture and stirring was continued for 3 hours at 800 rpm.

### Coating the Coating Composition

A polyethylene terephthalate film (75 μm thickness), which can function as a peelable film, was coated with the resulting asenapine-containing (in the form of its free base) coating composition and dried for approximately 10 minutes at room temperature and for 20 minutes at 80° C. The coating thickness gave a grammage of the matrix layer of 160.0 g/m². The resulting matrix layer has very good adhesive properties. The dried film was laminated with a polyethylene terephthalate back layer (23 μm thickness). The laminate was sealed in a packaging material (AR Neutral P/PET/AL/COC-Coex 628 mm) under nitrogen and stored at room temperature.

### Conversion of Asenapine Maleate and Purity of Asenapine in the Form of its Free Base

The conversion of asenapine maleate and the sum of the degradation products in Examples 5a-d were determined by means of quantitative HPLC. For this purpose, a liquid sample of 500 μl was taken by means of an Eppendorf pipette from the product mixtures of Examples 5a-d in each case after one day and after two days of reaction.

The following sample preparation steps and the following sample measurement methods were applied to all liquid samples of Examples 5a-d. For technical reasons, the sample preparations of the liquid samples differed slightly from one another; however without affecting the comparability of the results. A liquid sample was placed in a centrifuge and centrifuged therein for 15 minutes at room temperature and 15000 rpm. A 25 ml volumetric flask was charged with 2.0 ml of methanol (as extraction agent) for the centrifuged liquid sample. 25 μl of the centrifuged liquid sample was added using an Eppendorf pipette. This mixture was made up to volume using a phosphate buffer/methanol mixture (60:40 v/v, as diluent).

For the preparation of an HPLC standard, maleic acid was weighed out in a 20 ml volumetric flask and made up to volume with methanol and dissolved in order to obtain a stock solution with a concentration of 1031 μg/ml. 1.0 ml of this stock solution was pipetted into a 20 ml volumetric flask and made up to volume with a buffer solution in order to obtain a standard solution with a concentration of 51.55 μg/ml. The centrifuged and diluted liquid samples, which were taken out after one day, were measured with a dilution of 1:1000, whereas the centrifuged and diluted liquid samples, which were taken out after two days, were measured with a dilution of 1:2000 by means of quantitative HPLC. The quantitative HPLC measurement was carried out under the conditions detailed in the detailed description. The total mass of asenapine (containing asenapine in the form of its free base as well as asenapine maleate) and the mass of maleic acid were determined using the maleic acid standard solution.

For the determination of the conversion, in each case a total mass of asenapine (which originates from asenapine in the form of its free base as well as asenapine maleate) and a mass of the maleic acid of the liquid sample were determined by means of quantitative HPLC (see above). The mass fraction of asenapine in the form of its free base can thus be determined by calculation. In this case, the conversion of asenapine maleate (see Table 6.4) corresponds to the mass fraction of asenapine in the form of its free base based on the total mass of asenapine of the centrifuged and diluted liquid sample.

The liquid samples were also used to determine any degradation products of asenapine by means of quantitative HPLC. The smaller the sum of the degradation products, the higher the purity of asenapine in the form of its free base.

The conversions and the sum of the degradation products are summarized in Table 6.4.

TABLE 6.4

| | Conversion of asenapine | | Sum of the degradation | |
|---|---|---|---|---|
| | maleate | | products | |
| Ex. | Day 1 | Day 2 | Day 1 | Day 2 |
| Ex. 5a | 99.90% | 99.69% | <0.05% | <0.05% |
| Ex. 5b | 99.92% | 99.83% | <0.05% | <0.05% |
| Ex. 5c | 99.90% | 99.76% | <0.05% | 0.12% |
| Ex. 5d | 99.90% | 99.77% | <0.05% | <0.05% |

Furthermore, it was surprisingly found that a d50 particle diameter of sodium metasilicate of <125 μm resulted in a faster and higher conversion of asenapine maleate while high purities were achieved (see Table 6.4).

TABLE 6.5

| | Conversion of asenapine | Sum of the degradation |
|---|---|---|
| Ex. | maleate | products |
| Filtrate Ex. 5e | 99.9% | <0.05% |

The conversion of asenapine maleate as well as the sum of degradation products in Table 6.5 were determined using the quantitative HPLC analysis described above in detail.

### Analysis of the Matrix Layer

For the analysis of the mass fraction of asenapine in the form of its free base as well as the sum of the degradation products, asenapine was extracted from the matrix layer by means of methanol. The mass fraction of asenapine in the form of its free base as well as its purity are determined by means of quantitative HPLC.

TABLE 6.6

| Ingredient | Fraction [%] | |
|---|---|---|
| Asenapine in the form of its free base | 7.9 | |
| DURO-TAK 4287 | 71.1 | |
| Povidone K30 | 10.2 | |
| Miglyol 812 | 10.2 | |
| α-Tocopherol | 0.06 | |
| Ascorbyl palmitate | 0.2 | |
| Sodium metabisulfite | 0.109 | |
| Grammage | 160 | g/cm² |
| Mass fraction of asenapine in the form of its free base | 99.9% | |
| Sum of the degradation products | <0.05% | |
| Content of asenapine in the form of its free base | 1262.7 | μg/cm² |

From Table 6.5 it can be seen that a matrix layer of a TTS which contains asenapine in the form of its free base in high purity, could be obtained in a simple manner and without complex purification steps.

### Examples 6a-e

### Reaction Mixtures

The masses as well as the fractions of the ingredients of the reaction mixtures, which were used in Examples 6a-6e, are summarized in Tables 7.1 and 7.2. All percentages in Tables 7.1 and 7.2 refer to mass percent (% by mass).

TABLE 7.1

| | Ex. 6a | | Ex. 6b | | Ex. 6c | |
|---|---|---|---|---|---|---|
| | Mass | Fraction | Mass | Fraction | Mass | Fraction |
| Ingredient | [g] | [%] | [g] | [%] | [g] | [%] |
| Asenapine maleate | 0.5001 | 2.98 | 0.5001 | 2.92 | 0.4997 | 2.96 |
| Water (total)* | 0.0511 | 0.30 | 0.0449 | 0.26 | 0.0272 | 0.16 |
| Sodium metasilicate | 0.1533 | 0.91 | 0.3030 | 1.77 | 0.153 | 0.90 |
| Ethanol, absolute, | 16.0449 | 95.60 | 16.2425 | 94.93 | 16.2272 | 95.98 |
| ultrapure (total)** | | | | | | |
| α-Tocopherol | 0.0018 | 0.01 | 0.0032 | 0.02 | — | — |
| Ascorbyl palmitate | 0.0101 | 0.06 | 0.0108 | 0.06 | — | — |
| solution [10% in | | | | | | |
| ethanol absolute, | | | | | | |
| ultrapure]*** | | | | | | |
| Sodium metabisulfite | 0.0219 | 0.13 | 0.0055 | 0.03 | — | — |
| solution [30% in | | | | | | |
| water]**** | | | | | | |

*Sum of the masses/fractions of the water, which is added in the form of a 30% sodium metabisulfite solution, and the remaining water, which is added as part of the solvent of the reaction mixture.
**Sum of the masses/fractions of the ethanol, which is present in the form of 10% ascorbyl palmitate solution, and the remaining ethanol, which is added as part of the solvent of the reaction mixture.
***Mass and fraction refer to ascorbyl palmitate.
****Mass and fraction refer to sodium metabisulfite.

TABLE 7.2

| | Ex. 6d | | Ex. 6e | |
|---|---|---|---|---|
| | Mass | Fraction | Mass | Fraction |
| Ingredient | [g] | [%] | [g] | [%] |
| Asenapine maleate | 0.4998 | 2.93 | 3.5004 | 23.44 |
| Water (total)* | 0.0280 | 0.16 | 0.3456 | 2.31 |
| Sodium metasilicate | 0.3052 | 1.79 | 2.1337 | 14.29 |
| Ethanol, absolute, ultrapure | 16.2360 | 95.12 | 8.8120 | 59.01 |
| (total)** | | | | |
| α-Tocopherol | — | — | 0.0148 | 0.10 |
| Ascorbyl palmitate solution | — | — | 0.0804 | 0.54 |
| [10% in ethanol, | | | | |
| absolute, ultrapure]*** | | | | |
| Sodium metabisulfite solution | — | — | 0.0455 | 0.30 |
| [30% in water]**** | | | | |

*Sum of the masses/fractions of the water, which is added in the form of a 30% sodium metabisulfite solution, and the remaining water, which is added as part of the solvent of the reaction mixture.
**Sum of the masses/fractions of the ethanol, which is present in the form of 10% ascorbyl palmitate solution, and the remaining ethanol, which is added as part of the solvent of the reaction mixture.
***Mass and fraction refer to ascorbyl palmitate.
****Mass and fraction refer to sodium metabisulfite.

### Preparation of Asenapine in the Form of its Free Base

For providing the reaction mixtures according to Examples 6a-e, a reaction vessel was charged with α-tocopherol (apart from Examples 6c and 6d, in which no antioxidants were used). Thereafter, asenapine maleate was added. Ethanol (absolute, ultrapure) was added as a solvent, and the mixture was stirred at 300 rpm. Thereafter, a 30% aqueous sodium metabisulfite solution and a 10% ethanolic ascorbyl palmitate solution were added in succession with stirring (apart from Examples 6c and 6d, in which no antioxidants were used). Then, ground sodium metasilicate was added. Then, the mixture was flushed with argon. Thereafter, water as a further solvent is added to the mixture (apart from Example 6a, in which the water originates only from the 30% aqueous sodium metabisulfite solution), in order to obtain the reaction mixture, whereupon the reaction mixture was flushed again with argon. Then, the reaction mixture was stirred at 300 rpm for the conversion of asenapine maleate to asenapine in the form of its free base at approximately 20° C. The masses of the reaction mixtures according to Examples 6a-e are summarized in Tables 7.1 and 7.2.

### Working up the Reaction Mixture

After several days summarized in Tables 7.3 and 7.4, samples were taken from the reaction mixtures of Ex. 6a-e. A portion of the samples was filtered, followed by rinsing with ethanol (absolute, ultrapure) in order to obtain a filtrate. The remaining portion of the samples remained in the form of a suspension. The solvent of the samples (both in the form of filtrate as well as in the form of a suspension) was removed by means of a rotary evaporator (Rotavapor Büchi R-100, water bath temperature 50° C., pressure: 150 mbar). Then, the product was dried at 80° C. for 10 minutes and cooled in a desiccator. The product obtained in this way is called “solution” in Tables 7.3 and 7.4. Then, the total mass of the solution is determined using a scale. The solution is taken up in methanol and examined by means of quantitative HPLC with regard to the mass fraction of asenapine and the sum of the degradation products. The mass fraction of asenapine maleate with regard to the total mass of the solution is determined using the quantitative HPLC method. The sum of the degradation products was also determined by means of quantitative HPLC. Tables 7. 3 and 7.4 show the mass fractions of asenapine and the sum of the degradation products in the corresponding samples (solution, suspension, filtrate or centrifugate). Here, “suspension” is understood to mean the reaction mixture (without a filtration step). The sample preparation was done analogously to the liquid sample preparations mentioned above.

TABLE 7.3

| | Mass fraction of | Sum of the degradation |
|---|---|---|
| Example | asenapine | products |
| Ex. 6a | 73.1% | <0.05% |
| Solution after 2 days | | |
| Ex. 6b | 86.3% | <0.05% |
| Solution after 2 days | | |
| Ex. 6b | 92.3% | <0.05% |
| Solution after 5 days | | |
| Ex. 6c | 71.8% | 2.84% |
| Solution after 2 days | | |
| Ex. 6d | 77.3% | 0.78% |
| Solution after 1 day | | |
| Ex. 6d | 78.2% | 13.90% |
| Solution after 2 days | | |
| Ex. 6a | 103.6% | <0.05% |
| Suspension after 7 days | | |
| Ex. 6c | 105.1% | <0.05% |
| Suspension after 7 days | | |
| Ex. 6d | 106.8% | <0.05% |
| Suspension after 7 days | | |

From Table 7.3 it can be seen that in the solution with antioxidants (see solutions in Ex. 6a and 6b) less degradation products form, as compared to the solution without antioxidants (see solutions in Ex. 6c and 6d).

TABLE 7.4

| | Mass fraction of | Sum of the degradation |
|---|---|---|
| Example | asenapine | products |
| Ex. 6e | 80.4% | 0.29% |
| Solution after 2 days | | |
| Ex. 6e | 91.1% | 0.23% |
| Solution after 5 days | | |
| Ex. 6e | 91.0% | 0.34% |
| Solution after 8 days | | |
| Ex. 6e | 86.8% | 0.39% |
| Solution after 12 days | | |
| Ex. 6e | — | 0.27% |
| Centrifugate after 12 days | | |
| Ex. 6e | — | 0.27% |
| Filtrate after 12 days | | |

### THE INVENTION RELATES IN PARTICULAR TO THE FOLLOWING EMBODIMENTS

1. A process for the preparation of asenapine in the form of its free base comprising the steps of:
1) providing a reaction mixture comprising asenapine maleate and an alkali metal silicate in a solvent, wherein the reaction mixture contains the alkali metal silicate in dispersed form; and
2) reacting asenapine maleate with the alkali metal silicate in the reaction mixture provided in step 1) in order to obtain a product mixture which contains dissolved asenapine in the form of its free base and alkali metal maleate in dispersed form.

2. The process according to embodiment 1 further comprising the step of:
3) isolating a solution containing asenapine in the form of its free base from the product mixture obtained in step 2), wherein the isolation is preferably carried out by means of filtration.

3. The process according to embodiment 1 or 2, wherein the solvent used in step 1) contains water.

4. The process according to embodiment 3, wherein the solvent used in step 1) contains water and asenapine maleate in a molar ratio of 4 or less parts of water, preferably 3 or less parts of water, more preferably from 3 to ¼ parts of water, and most preferably from 2 to ⅓ parts of water, each based on 1 part of asenapine maleate.

5. The process according to any one of embodiments 1 to 4, wherein the alkali metal silicate is selected from the group consisting of sodium metasilicate, sodium trisilicate, potassium silicate and mixtures thereof.

6. The process according to any one of embodiments 1 to 5, wherein the d50 particle diameter of the alkali metal silicate used in step 1) is 125 μm or more, or less than 125 μm, or wherein the d80 particle diameter of the alkali metal silicate used in step 1) is less than 200 μm.

7. The process according to any one of embodiments 1 to 6, wherein the solvent used in step 1) contains alkali metal silicate and asenapine maleate in a molar ratio of 1 or more parts of alkali metal silicate, from 1 to 10 parts of alkali metal silicate, from 1 to 5 parts of alkali metal silicate, from 1 to 4 parts of alkali metal silicate, from 1 to 3 parts of alkali metal silicate, or from 1 to 2 parts of alkali metal silicate, each based on 1 part of asenapine maleate.

8. The process according to any one of embodiments 1 to 7, wherein the solvent in step 1) contains an alcoholic solvent which is preferably selected from the group consisting of methanol, ethanol, 1-propanol, 2-propanol and mixtures thereof.

9. The process according to embodiment 8, wherein the mass fraction of the alcoholic solvent is 40% by mass to 97% by mass, preferably 45% by mass to 70% by mass, based on the total mass of the reaction mixture provided in step 1).

10. The process according to any one of embodiments 1 to 9, wherein step 2) is carried out at a temperature of 15° C. to 45° C., at a temperature of 15° C. to 25° C., or at a temperature of 35° C. to 45° C.

11. The process according to any one of embodiments 1 to 10, wherein the mass fraction of the asenapine maleate is 1% by mass to 40% by mass, preferably 2% by mass to 35% by mass, particularly preferably 15% by mass to 30% by mass, based on the total mass of the reaction mixture provided in step 1).

12. The process according to any one of embodiments 1 to 11, wherein step 2) is carried out for a period of 2 h to 144 h, preferably for a period of 8 h to 60 h, particularly preferably for a period of 12 h to 50 h.

13. The process according to any one of embodiments 1 to 12, wherein the conversion in step 2) is 80% or more, preferably 90% or more, particularly preferably 99% or more, wherein the conversion is determined by means of HPLC.

14. The process according to any one of embodiments 1 to 13, wherein the asenapine in the form of its free base is formed in step 2) with a purity of 99.0% or more, preferably 99.5% or more, particularly preferably 99.95% or more, wherein the purity is determined by means of HPLC.

15. The process according to any one of embodiments 1 to 14, wherein antioxidants are used in step 1) as part of the reaction mixture, wherein the antioxidants are selected in particular from the group consisting of α-tocopherol, sodium metabisulfite, which is preferably used as an aqueous solution in step 1), ascorbyl palmitate, which is preferably used as an ethanolic solution in step 1), and mixtures thereof.

16. The process according to embodiment 15, wherein the mass fraction of α-tocopherol is 0.01% by mass to 0.5% by mass, based on the total mass of the reaction mixture provided in step 1); and/or

wherein the mass fraction of sodium metabisulfite is 0.01% by mass to 0.5% by mass, based on the total mass of the reaction mixture provided in step 1); and/or
wherein the mass fraction of ascorbyl palmitate is 0.05% by mass to 1.0% by mass, based on the total mass of the reaction mixture provided in step 1).

17. A process for the preparation of an active ingredient-containing layer for use in a transdermal therapeutic system, wherein the process comprises the following steps of:
i) preparing asenapine in the form of its free base by means of the process according to any one of embodiments 1 to 16;
ii) combining at least the asenapine in the form of its free base obtained in step i) and a polymer in a further solvent in order to obtain a coating composition, wherein the asenapine in the form of its free base obtained in step i) and used in step ii) is preferably contained in the solvent used in step ii), and is more preferably present in a solution isolated according to step 3) according to embodiment 2;
iii) coating the coating composition on a back layer, a peelable film or an intermediate film; and
iv) drying the coated coating composition to form the active ingredient-containing layer.

18. The process according to embodiment 17, wherein the solvent from step ii) is selected from alcoholic solvents, in particular methanol, ethanol, isopropanol and mixtures thereof, and from non-alcoholic solvents, in particular ethyl acetate, hexane, n-heptane, petroleum ether, toluene, and mixtures thereof, and wherein the solvent is particularly preferably selected from ethanol and ethyl acetate.

19. The process according to embodiment 17 or 18, wherein the polymer is an acrylic polymer and preferably a copolymer based on vinyl acetate, 2-ethylhexyl acrylate, 2-hydroxyethyl acrylate and glycidyl methacrylate, or based on vinyl acetate, 2-ethylhexyl acrylate, and 2-hydroxyethyl acrylate, which is provided as a solution, and is preferably present as a solution in ethyl acetate, n-heptane, methanol, ethanol and mixtures thereof with a solids content of 30% by mass to 60% by mass.

20. The process according to embodiment 19, wherein α-tocopherol, sodium metabisulfite, which is preferably provided in an aqueous solution, ascorbyl palmitate, which is preferably provided in an ethanolic solution, a triglyceride and/or a polyvinylpyrrolidone are combined with the isolated solution containing asenapine in the form of its free base and the acrylic polymer in the solvent in step ii) to obtain the coating composition.

21. The process according to embodiment 20, wherein the active ingredient-containing layer formed in step iv) comprises
A) 4% by mass to 12% by mass asenapine in the form of its free base; and
B) 65% by mass to 85% by mass acrylic polymer;
C) 5% by mass to 15% by mass polyvinylpyrrolidone;
D) 5% by mass to 15% by mass triglyceride;
E) 0.1% by mass to 0.5% by mass ascorbyl palmitate;
F) 0.05% by mass to 0.3% by mass sodium metabisulfite;
G) 0.01% by mass to 0.1% by mass α-tocopherol; and based on the total mass of the active ingredient-containing layer obtained in step iv) from the coating composition.

22. The process according to any one of embodiments 17 to 21, wherein the drying in step iv) is carried out at a temperature of 50° C. to 90° C., particularly preferably at a temperature of 60° C. to 85° C.

23. The process according to any one of embodiments 17 to 22, wherein the active ingredient-containing layer formed has a grammage in a range from 50 to 90 g/m² or 90 to 230 g/m².

24. The process according to any one of embodiments 17 to 23, wherein the asenapine in the form of its free base in the active ingredient-containing layer formed has a purity of 95.0% or more, preferably 99.0% or more, particularly preferably 99.95% or more, wherein the purity is determined by means of HPLC.

25. The process according to any one of embodiments 17 to 24, wherein the drying in step iv) is carried out for a period of 10 min to 60 min.

26. The process according to any one of embodiments 17 to 25, wherein a mass fraction of asenapine in the form of its free base is 95% by mass or more, preferably 99% by mass or more, particularly preferably 100% by mass, based on the asenapine total mass in the active ingredient-containing layer formed.

27. The process according to any one of embodiments 17 to 26, wherein the drying in step iv) is carried out in two stages, wherein the drying is carried out in the first stage at 15° C. to 25° C. for a period of 5 min to 15 min, followed by drying in the second stage at 60° C. to 85° C. for a period of 10 min to 40 min.

28. Asenapine in the form of its free base, obtainable by a process according to any one of embodiments 1 to 16, wherein the asenapine in the form of its free base has a purity of 99.0% or more, preferably 99.5% or more, particularly preferably 99.95% or more, wherein the purity is determined by means of HPLC, wherein the asenapine in the form of its free base is preferably present in a solvent and particularly preferably in a solution isolated according to step 3) according to embodiment 2.

29. An active ingredient-containing layer for use in a transdermal therapeutic system, obtainable by a process according to any one of embodiments 17 to 27, wherein the asenapine in the form of its free base in the active ingredient-containing layer has a purity of 99.0% or more, preferably 99.5% or more, particularly preferably 99.95% or more, wherein the purity is determined by means of HPLC.

30. A transdermal therapeutic system containing an active ingredient-containing layer according to embodiment 29.

1. A process for the preparation of asenapine in the form of its free base comprising the steps of:
1) providing a reaction mixture comprising asenapine maleate and an alkali metal silicate in a solvent, wherein the reaction mixture contains the alkali metal silicate in dispersed form, and
2) reacting asenapine maleate with the alkali metal silicate in the reaction mixture provided in step 1) in order to obtain a product mixture which contains dissolved asenapine in the form of its free base and alkali metal maleate in dispersed form.

2. The process according to claim 1 further comprising the step of:
3) isolating a solution containing asenapine in the form of its free base from the product mixture obtained in step 2.

3. The process according to claim 1, wherein the solvent used in step 1) contains water.

4. The process according to claim 1, wherein the alkali metal silicate is selected from the group consisting of sodium metasilicate, sodium trisilicate, potassium silicate and mixtures thereof.

5. The process according to claim 1, wherein the d50 particle diameter of the alkali metal silicate used in step 1) is 125 μm or more, or less than 125 μm.

6. The process according to claim 1, wherein the solvent in step 1) contains an alcoholic solvent.

7. A process for the preparation of an active ingredient-containing layer for use in a transdermal therapeutic system, wherein said process comprises the following steps of:
i. preparing asenapine in the form of its free base by means of the process according to claim 1,
ii. combining at least the asenapine in the form of its free base obtained in step i) and a polymer in a further solvent in order to obtain a coating composition;
iii. coating the coating composition on a back layer, a peelable film or an intermediate film, and
iv. drying the coated coating composition to form the active ingredient-containing layer.

8. Asenapine in the form of its free base, obtainable by a process according to claim 1, wherein the asenapine in the form of its free base has a purity of 99.0% or more, 99.5% or more, or 99.95% or more, wherein the purity is determined by means of HPLC.

9. An active ingredient-containing layer for use in a transdermal therapeutic system, obtainable by a process according to claim 7, wherein the asenapine in the form of its free base in the active ingredient-containing layer has a purity of 99.0 % or more, 99.5% or more, or more, wherein the purity is determined by means of HPLC.

10. A transdermal therapeutic system containing an active ingredient-containing layer according to claim 9.

11. The process according to claim 3, wherein the solvent used in step 1) contains water and asenapine maleate in a molar ratio of 4 or less parts of water, 3 or less parts of water, from 3 to ¼ parts of water, or from 2 to ⅓ parts of water, each based on 1 part of asenapine maleate.

12. The process according to claim 1, wherein the d80 particle diameter of the alkali metal silicate used in step 1) is less than 200 μm.

13. The process according to claim 6, wherein the alcoholic solvent is selected from the group consisting of methanol, ethanol, l-propanol, 2-propanol and mixtures thereof.

14. A process for the preparation of an active ingredient-containing layer for use in a transdermal therapeutic system, wherein said process comprises the following steps of:
i) preparing asenapine in the form of its free base by means of the process according to claim 2,
ii) combining at least the asenapine in the form of its free base obtained in step i) and a polymer in a further solvent in order to obtain a coating composition, wherein the asenapine in the form of its free base obtained in step i) and used in step ii) is present in a solution isolated according to step 3);
iii) coating the coating composition on a back layer, a peelable film or an intermediate film, and
iv) drying the coated coating composition to form the active ingredient-containing layer.

15. Asenapine in the form of its free base, obtainable by a process according to claim 2, wherein the asenapine in the form of its free base has a purity of 99.0% or more, 99.5% or more, or 99.95% or more, wherein the purity is determined by means of HPLC, and wherein the asenapine in the form of its free base is present in a solution isolated according to step 3).

16. An active ingredient-containing layer for use in a transdermal therapeutic system, obtainable by a process according to claim 14, wherein the asenapine in the form of its free base in the active ingredient-containing layer has a purity of 99.0 % or more, 99.5% or more, or 99.95% or more, wherein the purity is determined by means of HPLC.

17. A transdermal therapeutic system containing an active ingredient-containing layer according to claim 16.
